(19) United States
(12) Patent Application Publication
(10) Pub. No.: US 2019/0375723 A1
(43) Pub. Date: Dec. 12, 2019

(54) New Naphtho[2,3-B]Furan Derivatives

(71) Applicant: Boston Biomedical, Inc., Cambridge, MA (US)

(72) Inventors: Hitoshi Ban, Osaka-shi, Osaka (JP); Seiji Kamioka, Osaka-shi, Osaka (JP); Manabu Kusagi, Osaka-shi, Osaka (JP)

(21) Appl. No.: 16/463,162

(22) PCT Filed: Nov. 22, 2017
(86) PCT No.: PCT/IB2017/001573

§ 371 Date: May 22, 2019

Related U.S. Application Data
(60) Provisional application No. 62/425,315, filed on Nov. 22, 2016.

(51) Int. Cl. C07D 307/92

(52) U.S. Cl. CPC C07D 307/92; A61K 45/06

(57) ABSTRACT

The present invention provides a compound useful as a novel antitumor agent targeting a CSC that is important in continuous proliferation of malignant tumor, metastasis and recurrence of cancer, and its resistance to an antitumor agent; a medicament comprising the compound as an active ingredient; a pharmaceutical composition; and an antitumor agent; as well as a method of treating cancer and/or a method of preventing cancer. The present invention provides compounds represented by formula (I): or pharmaceutically acceptable salts thereof, wherein X is an oxygen atom or sulfur atom; R¹ is a hydrogen atom, an alkyl group, or the like; R² is a halogen atom or the like; R³ is a hydrogen atom, an alkyl group, or the like; m is 0, 1, 2, 3, or 4; and n is 1, 2, 3, or 4 (with the proviso that the sum of m and n is 1, 2, 3, or 4).

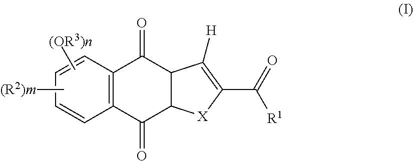

### TECHNICAL FIELD

The present invention relates to naphtho[2,3-b]furan derivatives useful as medicament and pharmaceutically acceptable salts thereof as well as antitumor agents comprising them as an active ingredient.

### BACKGROUND ART

Cancer develops when abnormality in gene occurs by an action of radiation, ultra violet rays, carcinogen, virus, and the like. The number of deaths by cancer increases year by year, and cancer is currently the top cause of death in Japan. As means for such cancer therapy, antitumor agents, surgical operation, radiotherapy, immunotherapy, and the like are performed. However, among these, the therapeutic use of an antitumor agent is the most important as an internally therapeutic means. Major antitumor agents act on any of metabolism of a nucleic acid precursor, DNA synthesis, RNA synthesis, or protein synthesis. However, such metabolic processes occur in not only cancer cells, but also normal cells. Therefore, many antitumor agents act on not only on cancer cells, but also on normal cells, and consequently a variety of side effects develop.

In recent years, a new type of antitumor agent, called molecular targeting agent, has been introduced. This molecular targeting agent is a pharmaceutical agent designed to target a molecule specifically expressed in each cancer. Therefore, it is believed that a molecular targeting agent has higher specificity to cancer cells than conventional antitumor agents, and has fewer side effects. However, with regard to molecular targeting agents, although previous side effects are reduced, there are problems that new side effects are exhibited and alternatives of pharmaceutical agents are limited. Although the aforementioned antitumor agents were clinically used for the purpose of cancer therapy and prolonging the life of a cancer patient, there are still a number of unsolved problems including problems of side effects and the like as described above. Accordingly, it is recognized that the development of a novel anticancer agent is an important object in the future.

In recent studies, the existence of cancer stem cells (CSC) having self-replication competence has been revealed. CSCs are identified in nearly all major types of cancer in humans, such as breast cancer, colon cancer, lung cancer, hematological malignancy, and the like, and are suggested to be closely related to continuous proliferation of malignant tumor, metastasis and recurrence of cancer, and its resistance to an antitumor agent (refer to Non Patent Literature 1). Since a CSC and a conventional cancer cell differentiated from the CSC are significantly different in biological characteristics, creation of an antitumor agent simultaneously targeting a CSC and a conventional cancer cell differentiated from the CSC is very promising as a new method to treat a cancer (refer to Non Patent Literature 2).

One of the characteristics of CSCs is to have replication competence (Refer to Non Patent Literature 3). Reliable methods established as a method of measuring replication competence of a cell include measurement of cancer cell sphere-forming ability in non-adhesion state in the absence of serum (Refer to Non Patent Literature 4). A compound that inhibits not only the proliferation of a non-CSC cancer cell, but also cancer cell sphere-forming ability is possibly very useful as a novel antitumor agent.

Previously, as naphtho[2,3-b]furan derivatives, for example, the following compounds described in Patent Literatures 1 to 6 and Non Patent Literatures 5 and 6 are already known.

(1) Patent Literature 1 describes the following compound having carcinostatic activity:

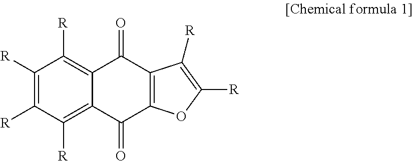

wherein R is —OR′, —R′, —CO—R′, and —OCO—R′, and R′ is a hydrogen atom, an alkyl group and/or a hydroxyalkyl group.

(2) Patent Literature 2 discloses the following compound:

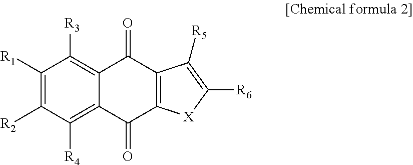

wherein X is —O—, —S—, or —NR₈—; R₁ and R₂ are, independently of each other, H, C₁-C₄alkyl, or the like; R₃ is a —OR₉ group; R₄ is H, halogen, or a —OR₉ group; R₅ and R₆ are, independently of each other, defined the same as R₁ and R₂, or R₅ and R₆ are taken together to form 1,2-benzobuta-1,3-diene-1,4-diyl; R₈ is H, linear or branched C₁-C₁₈alkyl, or each is phenyl or benzyl which is unsubstituted or substituted with C₁-C₄alkyl or C₁-C₄alkoxy; R₉ is C₆-C₁₄aryl which is unsubstituted or substituted with C₁-C₁₂alkyl, C₁-C₁₂alkoxy, C₁-C₁₂alkylthio, C₁-C₁₂alkylsulfinyl, C₁-C₁₂alkylsulfonyl, phenyl, benzyl, —CN, —CF₃, halogen, —CO—R₁₁, or —CO₂R₁₀, or the like.

(3) Patent Literature 3 describes the following compound having antitumor activity:

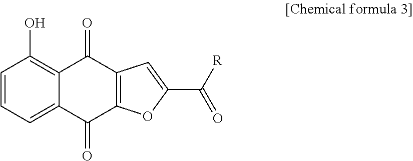

wherein R is C₁-C₆alkyl.

(4) Patent Literature 4 describes the following compound having antitumor activity and suppressing cancer cell sphere-forming ability:

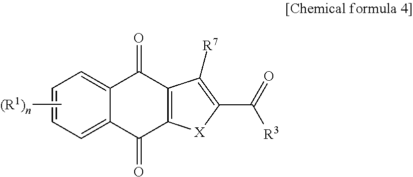

wherein X is O or S, R¹ is a hydrogen atom, a halogen atom, a cyano group, a nitro group, a trifluoromethyl group, a trifluoromethoxy group, an optionally substituted alkyl group, or the like, R³ is a hydrogen atom, a cyano group, an optionally substituted alkyl group, NR_{b}R_{c}, or the like, R_{b} and R_{c} are each independently a hydrogen atom, an alkyl group, or the like, R⁷ is a hydrogen atom, a halogen atom, a cyano group, a nitro group, an optionally substituted alkyl group, or the like, n is 1 to 4, with the proviso that when R³ is not NR_{b}R_{c}, R⁷ is not a hydrogen atom and at least one of R¹ and R⁷ is a halogen atom, an aryl group, or an optionally substituted aryl group.

(5) Patent Literature 5 discloses the following compound:

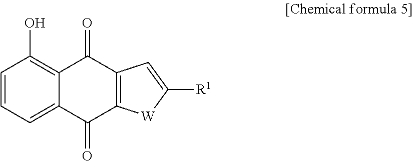

wherein R¹ is selected from the group consisting of a hydrogen atom; C₁₋₆alkyl optionally substituted with hydroxy, C₁₋₆alkoxy, or amino; C₅₋₁₀aryl optionally substituted with hydroxy, C₁₋₆alkoxy, or amino; a 5 to 10-membered saturated or unsaturated heterocycle optionally substituted with hydroxy, C₁₋₆alkoxy, or amino; CHO; CONH₂; C₁₋₆alkylcarbonyl optionally substituted with hydroxy, C₁₋₆alkoxy, or amino; and COC₅₋₁₀aryl optionally substituted with hydroxy, C₁₋₆alkoxy, or amino; W is O, S, or NR²; R² is selected from the group consisting of a hydrogen atom; C₁₋₆alkyl optionally substituted with nitro, sulfo, cyano, acetyl, or C₅₋₁₀aryl; COC₁₋₆alkyl; and COC₅₋₁₀aryl optionally substituted with nitro, sulfo, cyano, or acetyl.

(6) Patent Literature 6 discloses the following compound:

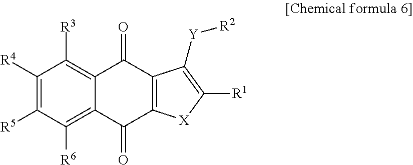

wherein X is an oxygen atom, a sulfur atom, or NR⁷; Y is —CO—, —CS—, or the like; R¹ is a hydrogen atom, a halogen atom, a cyano group, a nitro group, an optionally substituted C₁₋₆alkyl group, or the like; R² is a hydrogen atom, a hydroxy group, an optionally substituted C₁₋₆alkyl group, or the like; R³, R⁴, R⁵, and R⁶ are identical or different, and each independently a hydrogen atom, a halogen atom, a cyano group, a nitro group, a hydroxy group, an optionally substituted C₁₋₆alkyl group, or the like; and R⁷ is a hydrogen atom, an optionally substituted C₁₋₆alkyl group, an optionally substituted C₁₋₆alkylcarbonyl group, or the like.

(7) Non Patent Literature 5 discloses that the following quinone derivatives isolated from the extract of a plant of the Tabebuia genus in the Bignoniaceae family have antitumor activity:

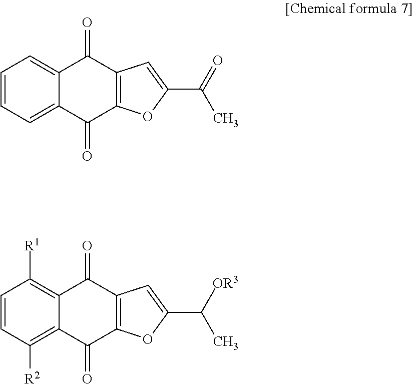

wherein R¹, R², and R³ are hydrogen atoms; R¹ and R³ are H, and R² is a hydroxyl group; R¹ is a hydroxyl group, and R² and R³ are hydrogen atoms; R¹ and R² are hydrogen atoms, and R³ is COCH₃; or R¹ and R² are hydrogen atoms, and R³ is COC (CF₃)(OCH₃)C₆H₅.

(8) Non Patent Literature 6 discloses the following compound:

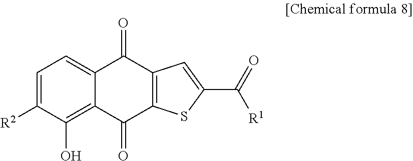

wherein R¹ is a hydrogen atom, a hydroxy group, an optionally substituted alkyl group, an optionally substituted alkoxy group, an optionally substituted amino group, an optionally substituted phenyl group, an optionally substituted heteroaryl group, or the like, R² is a hydrogen atom or an alkyl group optionally substituted with a hydroxy group, an ester group, an aryl group, a heteroaryl group, or the like.

None of Patent Literatures 1 to 6 and Non Patent Literatures 5 and 6, mentioned above, specifically discloses compounds represented by the following formula (I).

### CITATION LIST

### Patent Literature

[Patent Literature 1] Japanese Laid-Open Publication No. 63-196576
[Patent Literature 2] Japanese Laid-Open Publication No. 6-199787
[Patent Literature 3] International Publication No. 2006/098355
[Patent Literature 4] International Publication No. 2009/036059
[Patent Literature 5] Japanese Laid-Open Publication No. 2012-92083
[Patent Literature 6] International Publication No. 2015/120304

### Non Patent Literature

[Non Patent Literature 1] Boman et al., Journal of Clinical Oncology 26(17): 2795-2799. 2008
[Non Patent Literature 2] Lobo et al. Annu Rev Cell Dev Biol 23:675-99. 2007
[Non Patent Literature 3] Al-Hajj et al. Oncogene 23(43):7274-82. 2004
[Non Patent Literature 4] Ponti et al. Cancer Res 65(13):5506-11. 2005
[Non Patent Literature 5] Rao et al. J Nat Prod 45(5):600-4. 1982
[Non Patent Literature 6] Bannwitz et al. J Med Chem 57:6226-6239. 2014

### SUMMARY OF INVENTION

### Solution to Problem

The present invention provides a compound useful as a novel antitumor agent targeting a CSC that is important in continuous proliferation of malignant tumor, metastasis and recurrence of cancer, and its resistance to an antitumor agent; a medicament comprising the compound as an active ingredient; a pharmaceutical composition; and an antitumor agent; as well as a method of treating cancer and/or a method of preventing cancer.

The present inventors focused on quinone derivatives, and intensively studied with regard to their antitumor activities to find that a compound represented by the following formula (I) or a pharmaceutically acceptable salt thereof (hereinafter also referred to as “the present compound”) is useful as a novel antitumor agent targeting a CSC that is important in continuous proliferation of malignant tumor, metastasis and recurrence of cancer, and its resistance to an antitumor agent. The present inventors finally reached the completion of the present invention.

Specifically, the present invention is as described below.

Item 1: A compound represented by formula (I):

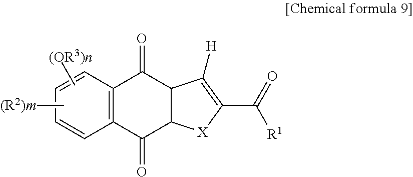

or a pharmaceutically acceptable salt thereof, wherein
X is an oxygen atom or sulfur atom;
R¹ is a hydrogen atom, a cyano group, a trifluoromethyl group, a trifluoromethoxy group, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, an optionally substituted 5 to 12-membered monocyclic or polycyclic heteroaryl group, —OR⁴, or —SR⁴;
each R² is independently a halogen atom, a cyano group, a nitro group, a trifluoromethyl group, a trifluoromethoxy group, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted C₁₋₆alkylcarbonyl group, an optionally substituted C₃₋₁₀cycloalkylcarbonyl group, an optionally substituted C₆₋₁₀arylcarbonyl group, an optionally substituted 5 to 12-membered monocyclic or polycyclic heteroarylcarbonyl group, an optionally substituted 3 to 10-membered saturated heterocyclic group, an optionally substituted C₆₋₁₀aryl group, an optionally substituted 5 to 12-membered monocyclic or polycyclic heteroaryl group, an optionally substituted C₁₋₆alkylsulfonyl group, an optionally substituted C₃₋₁₀cycloalkylsulfonyl group, an optionally substituted C₆₋₁₀arylsulfonyl group, an optionally substituted aminosulfonyl group, —N(R⁴)₂, or —SR⁴;
each R³ is independently a hydrogen atom, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, an optionally substituted 5 to 12-membered monocyclic or polycyclic heteroaryl group, an optionally substituted C₁₋₆alkylcarbonyl group, an optionally substituted C₃₋₁₀cycloalkylcarbonyl group, an optionally substituted C₆₋₁₀arylcarbonyl group, an optionally substituted 3 to 10-membered saturated heterocyclylcarbonyl group, an optionally substituted 5 to 12-membered monocyclic or polycyclic heteroarylcarbonyl group, an optionally substituted C₁₋₆alkyloxycarbonyl group, an optionally substituted C₃₋₁₀cycloalkyloxycarbonyl group, an optionally substituted C₆₋₁₀aryloxycarbonyl group, an optionally substituted 3 to 10-membered saturated heterocyclyloxycarbonyl group, an optionally substituted 5 to 12-membered monocyclic or polycyclic heteroaryloxycarbonyl group, an optionally substituted C₁₋₆alkylaminocarbonyl group, an optionally substituted C₃₋₁₀cycloalkylaminocarbonyl group, an optionally substituted C₆₋₁₀arylaminocarbonyl group, an optionally substituted 3 to 10-membered saturated heterocyclylaminocarbonyl group, or an optionally substituted 5 to 12-membered monocyclic or polycyclic heteroarylaminocarbonyl group;
m is 0, 1, 2, 3, or 4 and n is 1, 2, 3, or 4, with the proviso that the sum of m and n is 1, 2, 3, or 4; and
each R⁴ is independently a hydrogen atom, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, or an optionally substituted 5 to 12-membered monocyclic or polycyclic heteroaryl group, or when two R⁴ are attached to a nitrogen atom, the two R⁴ may be taken together with the nitrogen atom to which they are attached to form a nitrogen-containing 3 to 10-membered saturated heterocyclic group,
with the proviso that the following compounds are excluded:
5-hydroxy-4,9-dioxo-4,9-dihydronaphtho[2,3-b]furan-2-carbaldehyde;
2-acetyl-5-hydroxynaphtho[2,3-b]furan-4,9-dione;
2-acetyl-8-hydroxynaphtho[2,3-b]furan-4,9-dione;
2-acetyl-6-methoxynaphtho[2,3-b]furan-4,9-dione;
2-acetyl-7-methoxynaphtho[2,3-b]furan-4,9-dione;
2-acetyl-8-methoxynaphtho[2,3-b]furan-4,9-dione;
2-acetyl-5-methoxynaphtho[2,3-b]furan-4,9-dione;
2-acetyl-8-hydroxy-7-methoxynaphtho[2,3-b]furan-4,9-dione;
2-acetyl-6,7-dimethoxynaphtho[2,3-b]furan-4,9-dione;
2-acetyl-7,8-dimethoxynaphtho[2,3-b]furan-4,9-dione;
2-acetyl-5,6-dimethoxynaphtho[2,3-b]furan-4,9-dione;
2-benzoyl-5-hydroxynaphtho[2,3-b]furan-4,9-dione;
8-hydroxy-4,9-dioxo-4,9-dihydronaphtho[2,3-b]thiophene-2-carbaldehyde;
5-hydroxy-4,9-dioxo-4,9-dihydronaphtho[2,3-b]thiophene-2-carbaldehyde;
8-methoxy-4,9-dioxo-4,9-dihydronaphtho[2,3-b]thiophene-2-carbaldehyde;
8-hydroxy-4,9-dioxo-4,9-dihydronaphtho[2,3-b]thiophene-2-carboxylic acid;
2-acetyl-8-hydroxynaphtho[2,3-b]thiophene-4,9-dione;
2-acetyl-5-hydroxynaphtho[2,3-b]thiophene-4,9-dione;
2-acetyl-5,8-dihydroxynaphtho[2,3-b]thiophene-4,9-dione;
8-hydroxy-2-propanoylnaphtho[2,3-b]thiophene-4,9-dione;
2-acetyl-8-methoxynaphtho[2,3-b]thiophene-4,9-dione;
8-methoxy-4,9-dioxo-4,9-dihydronaphtho[2,3-b]thiophene-2-carboxylic acid;
methyl 8-hydroxy-4,9-dioxo-4,9-dihydronaphtho[2,3-b]thiophene-2-carboxylate;
methyl 5-hydroxy-4,9-dioxo-4,9-dihydronaphtho[2,3-b]thiophene-2-carboxylate;
8-hydroxy-2-(2-methylpropanoyl)naphtho[2,3-b]thiophene-4,9-dione;
2-acetyl-8-hydroxy-7-(hydroxymethyl)naphtho[2,3-b]thiophene-4,9-dione;
2-acetyl-5,8-dimethoxynaphtho[2,3-b]thiophene-4,9-dione;
8-methoxy-2-propanoylnaphtho[2,3-b]thiophene-4,9-dione;
ethyl 8-hydroxy-4,9-dioxo-4,9-dihydronaphtho[2,3-b]thiophene-2-carboxylate;
8-hydroxy-2-(thiophene-2-ylcarbonyl)naphtho[2,3-b]thiophene-4,9-dione;
8-methoxy-2-(2-methylpropanoyl)naphtho[2,3-b]thiophene-4,9-dione;
2-benzoyl-5-hydroxynaphtho[2,3-b]thiophene-4,9-dione;
2-benzoyl-8-hydroxynaphtho[2,3-b]thiophene-4,9-dione;
propan-2-yl 8-hydroxy-4,9-dioxo-4,9-dihydronaphtho[2,3-b]thiophene-2-carboxylate;
ethyl 8-methoxy-4,9-dioxo-4,9-dihydronaphtho[2,3-b]thiophene-2-carboxylate;
8-methoxy-2-(thiophene-2-ylcarbonyl)naphtho[2,3-b]thiophene-4,9-dione;
propan-2-yl 8-methoxy-4,9-dioxo-4,9-dihydronaphtho[2,3-b]thiophene-2-carboxylate;
2-benzoyl-8-methoxynaphtho[2,3-b]thiophene-4,9-dione;
8-hydroxy-2-(phenyl acetyl)naphtho[2,3-b]thiophene-4,9-dione;
8-hydroxy-2-(4-methoxybenzoyl)naphtho[2,3-b]thiophene-4,9-dione;
2-(4-chlorobenzoyl)-8-hydroxynaphtho[2,3-b]thiophen-4,9-dione;
8-methoxy-2-(4-methoxybenzoyl)naphtho[2,3-b]thiophene-4,9-dione;
8-hydroxy-2-(3-phenylpropanoyl)naphtho[2,3-b]thiophene-4,9-dione;
8-methoxy-2-(phenylacetyl)naphtho[2,3-b]thiophene-4,9-dione;
8-methoxy-2-(3-phenylpropanoyl)naphtho[2,3-b]thiophene-4,9-dione;
8-hydroxy-2-(phenoxyacetyl)naphtho[2,3-b]thiophene-4,9-dione;
8-hydroxy-2-(pyridin-3-ylcarbonyl)naphtho[2,3-b]thiophene-4,9-dione;
2-(4-chlorobenzoyl)-8-methoxynaphtho[2,3-b]thiophene-4,9-dione;
4-methoxybenzyl 8-hydroxy-4,9-dioxo-4,9-dihydronaphtho[2,3-b]thiophene-2-carboxylate;
2-phenylethyl 8-hydroxy-4,9-dioxo-4,9-dihydronaphtho[2,3-b]thiophene-2-carboxylate;
8-methoxy-2-(phenoxyacetyl)naphtho[2,3-b]thiophene-4,9-dione;
8-methoxy-2-(pyridin-3-ylcarbonyl)naphtho[2,3-b]thiophene-4,9-dione;
4-methoxybenzyl 8-methoxy-4,9-dioxo-4,9-dihydronaphtho[2,3-b]thiophene-2-carboxylate;
8-hydroxy-2-(2-hydroxy-4-methoxybenzoyl)naphtho[2,3-b]thiophene-4,9-dione;
2-phenylethyl 8-methoxy-4,9-dioxo-4,9-dihydronaphtho[2,3-b]thiophene-2-carboxylate; and
2-(2-hydroxy-4-methoxybenzoyl)-8-methoxynaphtho[2,3-b]thiophene-4,9-dione.

Item 2: The compound according to item 1, or a pharmaceutically acceptable salt thereof, wherein from the compound, the following is excluded:

a compound in which X is a sulfur atom, m is 0, n is 1, R¹ is a hydrogen atom, a hydroxyl group, a methyl group, an ethyl group, a methoxy group, an ethoxy group, a phenyl group, or a group represented by the following:

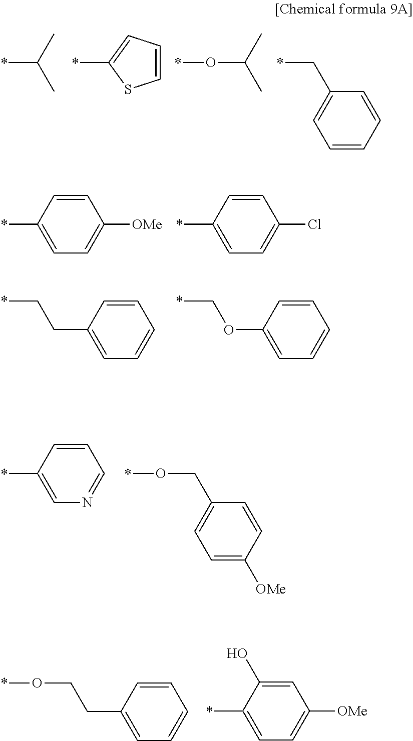

(wherein * is the point of attachment in the above)
and R³ is hydrogen;

a compound in which X is a sulfur atom, m is 0, n is 1, R¹ is a hydrogen atom, a hydroxyl group, a methyl group, an ethyl group, an ethoxy group, a phenyl group, or a group represented by the following:

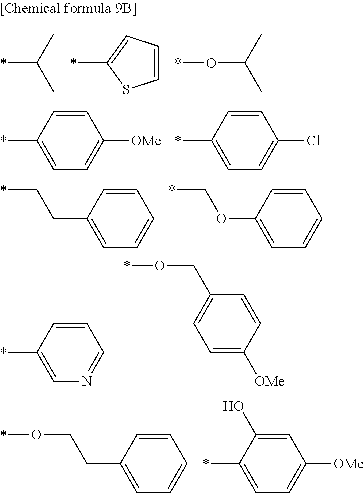

(wherein * is the point of attachment in the above)
and R³ is a methyl group; and
2-acetyl-5,8-dihydroxynaphtho[2,3-b]thiophene-4,9-dione;
2-acetyl-8-hydroxy-7-(hydroxymethyl)naphtho[2,3-b]thiophene-4,9-dione;
2-acetyl-5,8-dimethoxynaphtho[2,3-b]thiophene-4,9-dione;
5-hydroxy-4,9-dioxo-4,9-dihydronaphtho[2,3-b]furan-2-carbaldehyde;
2-acetyl-5-hydroxynaphtho[2,3-b]furan-4,9-dione;
2-acetyl-8-hydroxynaphtho[2,3-b]furan-4,9-dione;
2-acetyl-6-methoxynaphtho[2,3-b]furan-4,9-dione;
2-acetyl-7-methoxynaphtho[2,3-b]furan-4,9-dione,
2-actyl-8-methoxynaphtho[2,3-b]furan-4,9-dione;
2-acetyl-5-methoxynaphtho[2,3-b]furan-4,9-dione;
2-acetyl-8-hydroxy-7-methoxynaphtho[2,3-b]furan-4,9-dione;
2-acetyl-6,7-dimethoxynaphtho[2,3-b]furan-4,9-dione;
2-acetyl-7,8-dimethoxynaphtho[2,3-b]furan-4,9-dione;
2-acetyl-5,6-dimethoxynaphtho[2,3-b]furan-4,9-dione; and
2-benzoyl-5-hydroxynaphtho[2,3-b]furan-4,9-dione.

Item 3: The compound according to item 1 or 2, or a pharmaceutically acceptable salt thereof, wherein X is an oxygen atom.

Item 4: The compound according to any one of items 1 to 3, or a pharmaceutically acceptable salt thereof, wherein R¹ is a C₁₋₆alkyl group optionally substituted with one to three halogen atoms, hydroxyl groups, one or two C₁₋₆alkyloxy groups, or a 3 to 10-membered saturated heterocyclic group.

Item 5: The compound according to item 4, or a pharmaceutically acceptable salt thereof, wherein R¹ is a methyl group.

Item 6: The compound according to any one of items 1 to 5, or a pharmaceutically acceptable salt thereof, wherein each R² is independently a halogen atom, a cyano group, an optionally substituted C₁₋₆alkyl group, an optionally substituted C₂₋₆alkenyl group, an optionally substituted C₂₋₆alkynyl group, an optionally substituted C₃₋₁₀cycloalkenyl group, an optionally substituted C₁₋₆alkylcarbonyl group, an optionally substituted C₆₋₁₀aryl group, or an optionally substituted 5 to 12-membered monocyclic or polycyclic heteroaryl group.

Item 7: The compound according to item 6, or a pharmaceutically acceptable salt thereof, wherein each R² is independently a halogen atom, an optionally substituted C₁₋₆alkyl group, an optionally substituted C₆₋₁₀aryl group; or an optionally substituted 5 to 12-membered monocyclic or polycyclic heteroaryl group.

Item 8: The compound according to item 6 or 7, or a pharmaceutically acceptable salt thereof, wherein each R² is independently a halogen atom, or an optionally substituted C₁₋₆alkyl group.

Item 9: The compound according to any one of items 6 to 8, or a pharmaceutically acceptable salt thereof, wherein each R² is independently a halogen atom.

Item 10: The compound according to any one of items 1 to 9, or a pharmaceutically acceptable salt thereof, wherein each R³ is independently a hydrogen atom, an optionally substituted C₁₋₆alkyl group, an optionally substituted C₆₋₁₀aryl group, an optionally substituted 5 to 12-membered monocyclic or polycyclic heteroaryl group, an optionally substituted C₁₋₆alkylcarbonyl group, an optionally substituted C₆₋₁₀arylcarbonyl group, an optionally substituted C₁₋₆alkyloxycarbonyl group, an optionally substituted C₁₋₆aryloxycarbonyl group, an optionally substituted C₁₋₆alkylaminocarbonyl group, or an optionally substituted C₆₋₁₀arylaminocarbonyl group.

Item 11: The compound according to item 10, or a pharmaceutically acceptable salt thereof, wherein each R³ is independently a hydrogen atom, an optionally substituted C₁₋₆alkyl group, an optionally substituted C₆₋₁₀arylcarbonyl group, or an optionally substituted C₁₋₆alkyloxycarbonyl group.

Item 12: The compound according to item 10 or 11, or a pharmaceutically acceptable salt thereof, wherein each R³ is independently a hydrogen atom.

Item 13: The compound according to any one of items 1 to 12, or a pharmaceutically acceptable salt thereof, wherein each R⁴ is independently a hydrogen atom, an optionally substituted C₁₋₆alkyl group, an optionally substituted C₃₋₁₀cycloalkyl group, an optionally substituted C₆₋₁₀aryl group, or an optionally substituted 5 to 12-membered monocyclic or polycyclic heteroaryl group, or when two R⁴ are attached to a nitrogen atom, the two R⁴ may be taken together with the nitrogen atom to which they are attached to form a nitrogen-containing 3 to 10-membered saturated heterocyclic group.

Item 14: The compound according to item 13, or a pharmaceutically acceptable salt thereof, wherein each R⁴ is independently a hydrogen atom or an optionally substituted C₁₋₆alkyl group, or when two R⁴ are attached to a nitrogen atom, the two R⁴ may be taken together with the nitrogen atom to which they are attached to form a nitrogen-containing 3 to 10-membered saturated heterocyclic group.

Item 15: The compound according to any one of items 1 to 14, or a pharmaceutically acceptable salt thereof, wherein m and n are both 1.

Item 16: The compound according to item 1 or 2, or a pharmaceutically acceptable salt thereof, wherein the compound is selected from the group consisting of the following compounds:
2-acetoxy-8-chloro-5-hydroxynaphtho[2,3-b]furan-4,9-dione;
2-acetyl-6-bromo-5-hydroxynaphtho[2,3-b]furan-4,9-dione;
2-acetyl-7-bromo-8-hydroxynaphtho[2,3-b]furan-4,9-dione;
2-acetyl-6-butyl-5-hydroxynaphtho[2,3-b]furan-4,9-dion;
2-acetyl-5-hydroxy-6-(4-methoxyphenyl)naphtho[2,3-b]furan-4,9-dione;
2-acetyl-5-hydroxy-6-(2-methoxypyrimidin-5-yl)naphtho[2,3-b]furan-4,9-dione;
2-acetyl-6-bromo-5-(methoxymethoxy)naphtho[2,3-b]furan-4,9-dione;
2-acetyl-6-bromo-4,9-dioxo-4,9-dihydronaphtho[2,3-b]furan-5-yl benzoate;
2-acetyl-6-bromo-4,9-dioxo-4,9-dihydronaphtho[2,3-b]furan-5-yl 2-methylpropyl carbonate;
2-acetyl-6-chloro-5-hydroxynaphtho[2,3-b]furan-4,9-dione;
2-acetyl-7-chloro-5-hydroxynaphtho[2,3-b]furan-4,9-dione;
2-acetyl-5-chloro-8-hydroxynaphtho[2,3-b]furan-4,9-dione;
2-acetyl-6-chloro-8-hydroxynaphtho[2,3-b]furan-4,9-dione;
2-acetyl-7-chloro-8-hydroxynaphtho[2,3-b]furan-4,9-dione;
2-acetyl-8-chloro-7-hydroxynaphtho[2,3-b]furan-4,9-dione;
2-acetyl-6-chloro-7-hydroxynaphtho[2,3-b]furan-4,9-dione;
2-acetyl-5-chloro-7-hydroxynaphtho[2,3-b]furan-4,9-dione;
2-acetyl-5-chloro-6-hydroxynaphtho[2,3-b]furan-4,9-dione;
2-acetyl-7-chloro-6-hydroxynaphtho[2,3-b]furan-4,9-dione; and
2-acetyl-8-chloro-6-hydroxynaphtho[2,3-b]furan-4,9-dione.

Item 17: A pharmaceutical composition comprising a compound according to any one of items 1 to 16 or a pharmaceutically acceptable salt thereof.

Item 18: An anticancer agent comprising a compound according to any one of items 1 to 16 or a pharmaceutically acceptable salt thereof as an active ingredient.

Item 19: The anticancer agent according to item 18, wherein the cancer is at least one cancer selected from the group consisting of acute leukemia, chronic lymphocytic leukemia, chronic myelocytic leukemia, polycythemia vera, malignant lymphoma, brain tumor, head and neck cancer, esophageal cancer, thyroid cancer, small cell lung cancer, non-small-cell lung cancer, breast cancer, gastric cancer, gallbladder⋅bile duct cancer, hepatoma, pancreatic cancer, colon cancer, rectal cancer, chorioepithelioma, chorioblastoma, choriocarcinoma, endometrial cancer, cervical cancer, urothelial cancer, renal cell cancer, testicular tumor, Wilms' tumor, skin cancer, malignant melanoma, neuroblastoma, osteosarcoma, Ewing's sarcoma, and soft tissue sarcoma.

Item 20: A method for treating a cancer, characterized by administering a therapeutically effective amount of a compound according to any one of items 1 to 16 or a pharmaceutically acceptable salt thereof to a patient in need of the treatment.

Item 21: The method for treating according to item 20, wherein the cancer is at least one cancer selected from the group consisting of acute leukemia, chronic lymphocytic leukemia, chronic myelocytic leukemia, polycythemia vera, malignant lymphoma, brain tumor, head and neck cancer, esophageal cancer, thyroid cancer, small cell lung cancer, non-small-cell lung cancer, breast cancer, gastric cancer, gallbladder⋅bile duct cancer, hepatoma, pancreatic cancer, colon cancer, rectal cancer, chorioepithelioma, chorioblastoma, choriocarcinoma, endometrial cancer, cervical cancer, urothelial cancer, renal cell cancer, testicular tumor, Wilms' tumor, skin cancer, malignant melanoma, neuroblastoma, osteosarcoma, Ewing's sarcoma, and soft tissue sarcoma.

Item 22: The use of a compound according to any one of items 1 to 16 or a pharmaceutically acceptable salt thereof for the manufacture of a therapeutic agent for cancer.

Item 23: The use of a compound according to item 22 or a pharmaceutically acceptable salt thereof, wherein the cancer is at least one cancer selected from the group consisting of acute leukemia, chronic lymphocytic leukemia, chronic myelocytic leukemia, polycythemia vera, malignant lymphoma, brain tumor, head and neck cancer, esophageal cancer, thyroid cancer, small cell lung cancer, non-small-cell lung cancer, breast cancer, gastric cancer, gallbladder⋅bile duct cancer, hepatoma, pancreatic cancer, colon cancer, rectal cancer, chorioepithelioma, chorioblastoma, choriocarcinoma, endometrial cancer, cervical cancer, urothelial cancer, renal cell cancer, testicular tumor, Wilms' tumor, skin cancer, malignant melanoma, neuroblastoma, osteosarcoma, Ewing's sarcoma, and soft tissue sarcoma.

In the present invention, it is intended that one or a plurality of the above-described characteristics can be further combined and provided in addition to clearly expressed combinations. Still further embodiments and advantages of the present invention will be recognized by those skilled in the art if the following detailed descriptions are read and understood as necessary.

### Advantageous Effects of Invention

Compounds represented by formula (I) or pharmaceutically acceptable salts thereof are useful as a novel antitumor agent targeting a CSC that is important in continuous proliferation of malignant tumor, metastasis and recurrence of cancer, and its resistance to an antitumor agent.

### DESCRIPTION OF EMBODIMENTS

Hereinafter, the present invention is described while showing the most preferable embodiment. Throughout the entire specification, a singular expression should be understood as encompassing the concept thereof in the plural form, unless specifically noted otherwise. Thus, singular expressions (e.g., “a”, “an”, “the” and the like in case of English) should also be understood as encompassing the concept thereof in the plural form unless specifically noted otherwise. Further, the terms used herein should be understood as being used in the meaning that is commonly used in the art, unless specifically noted otherwise. Thus, unless defined otherwise, all terminologies and scientific technical terms that are used herein have the same meaning as the terms commonly understood by those skilled in the art to which the present invention pertains. In case of a contradiction, the present specification (including the definitions) takes precedence.

### Definition

In the present specification, the number of substituents of a group defined by “optionally substituted” or “substituted” is not particularly limited if it is substitutable, and is one or plural. In addition, unless indicated otherwise, the description for each group is also applied when the group is one part of or a substituent on other groups.

In the present specification, examples of “halogen atom” include a fluorine atom, a chlorine atom, a bromine atom, and an iodine atom. Preferably, it is a fluorine atom or a chlorine atom.

A “C₁₋₆alkyl group” refers to a linear or branched, saturated hydrocarbon group of which the carbon number is one to six. Preferably, it includes a “C₁₋₄ alkyl group” and the like. Specific examples of the “C₁₋₆alkyl group” include, for example, a methyl group, an ethyl group, a propyl group, a 1-methylethyl group, a butyl group, a 2-methylpropyl group, a 1-methylpropyl group, a 1,1-dimethylethyl group, a pentyl group, a 3-methylbutyl group, a 2-methylbutyl group, a 2,2-dimethylpropyl group, a 1-ethylpropyl group, a 1,1-dimethylpropyl group, a hexyl group, a 4-methylpentyl group, a 3-methylpentyl group, a 2-methylpentyl group, a 1-methylpentyl group, and the like. Specific examples of the “C₁₋₆ alkyl group” include those having a carbon number from 1 to 4 that are exemplified in the specific examples of “C₁₋₆alkyl group”.

In the specification, for example, C₁₋₆ represents that the carbon number is one to six, C₁₋₄ represents that the carbon number is one to four, and C₆ represents that the carbon number is six. The same applies to cases of other numbers.

A “C₂₋₆ alkenyl group” refers to a linear or branched, unsaturated hydrocarbon group that has two to six carbons and contains one to three double bonds. Specific examples of the “C₂₋₆ alkenyl group” include, for example, a vinyl group, a propenyl group, a methylpropenyl group, a butenyl group, a methylbutenyl group, a pentenyl group, a hexenyl group, and the like.

A “C₂₋₆ alkynyl group” refers to a linear or branched, unsaturated hydrocarbon group that has two to six carbons and contains one triple bond. Specific examples of the “C₂₋₆ alkynyl group” include, for example, a propynyl group, a methylpropynyl group, a butynyl group, a methylbutynyl group, a pentynyl group, a hexynyl group, and the like.

A “C₃₋₁₀ cycloalkyl group” refers to a cyclic saturated hydrocarbon group of which the carbon number is three to ten, and also includes those having a bridged structure. Preferably, it includes a “C₃₋₇ cycloalkyl group” and the like. Specific examples of the “C₃₋₁₀ cycloalkyl group” include, for example, a cyclopropyl group, a cyclobutyl group, a cyclopentyl group, a cyclohexyl group, a cycloheptyl group, a cyclooctyl group, a cyclononyl group, a cyclodecyl group, an adamantyl group, and the like. Specific examples of the “C₃₋₇ cycloalkyl group” include, for example, a cyclopropyl group, a cyclobutyl group, a cyclopentyl group, a cyclohexyl group, a cycloheptyl group, and the like. The “C₃₋₁₀ cycloalkyl group” also encompasses a compound fused to an aromatic hydrocarbon ring. Specific examples of such a ring-fused compound include, for example, groups represented by the following:

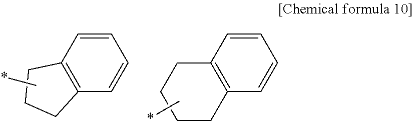

(wherein * is the point of attachment in the above)
and the like.

A “C₃₋₁₀ cycloalkenyl group” refers to a cyclic unsaturated hydrocarbon group that has three to ten carbons and contains one to three double bonds, and also includes those having a bridged structure. Preferably, it includes “C₃₋₇ cycloalkenyl group” and the like. Specific examples of the “C₃₋₁₀ cycloalkenyl group” include, for example, a cyclopropenyl group, a methylcyclopropenyl group, a cyclobutenyl group, a methylcyclobutenyl group, a cyclopentenyl group, a cyclohexenyl group, and the like.

A “C₁₋₆alkyloxy group” refers to an oxy group substituted with the above-described “C₁₋₆alkyl”, and is preferably a “C₁₋₄alkyloxy group” or the like. Specific examples of the “C₁₋₆alkyloxy group” include, for example, a methoxy group, an ethoxy group, a propoxy group, a 1-methylethoxy group, a butoxy group, a 2-methylpropoxy group, a 1-methylpropoxy group, a 1,1-dimethylethoxy group, a pentyloxy group, a 3-methylbutoxy group, a 2-methylbutoxy group, a 2,2-dimethylpropoxy group, a 1-ethylpropoxy group, a 1,1-dimethylpropoxy group, a hexyloxy group, a 4-methylpentyloxy group, a 3-methylpentyloxy group, a 2-methylpentyloxy group, a 1-methylpentyloxy group, a 3,3-dimethylbutoxy group, a 2,2-dimethylbutoxy group, a 1,1-dimethylbutoxy group, a 1,2-dimethylbutoxy group, and the like. In the relevant field, an “alkyloxy group” is sometimes referred to as an “alkoxy group”, however, both are synonymous.

A “C₃₋₁₀ cycloalkyloxy group” refers to an oxy group substituted with the above-described “C₃₋₁₀ cycloalkyl”, and is preferably a “C₃₋₇ cycloalkyloxy group”. Specific examples of the “C₃₋₁₀ cycloalkyloxy group” include, for example, a cyclopropoxy group, a cyclobutoxy group, a cyclopentyloxy group, a cyclohexyloxy group, a cycloheptyloxy group, a cyclooctyloxy group, a cyclononyloxy group, a cyclodecyloxy group, an adamantyloxy group, and the like. In the relevant field, a “cycloalkyloxy group” is sometimes referred to as a “cycloalkoxy group”, however, both are synonymous.

A “C₁₋₆alkylcarbonyl group” refers to a carbonyl group substituted with the above-described “C₁₋₆alkyl”, and is preferably a “C₁₋₄alkylcarbonyl group”. Specific examples of the “C₁₋₆alkylcarbonyl group” include, for example, a methylcarbonyl group, an ethylcarbonyl group, a propylcarbonyl group, a 1-methylethylcarbonyl group, a butylcarbonyl group, a 2-methylpropylcarbonyl group, a 1-methylpropylcarbonyl group, a 1,1-dimethylethylcarbonyl group, and the like.

A “C₃₋₁₀cycloalkylcarbonyl group” refers to a carbonyl group substituted with the above-described “C₃₋₁₀cycloalkyl”, and is preferably a “C₃₋₇cycloalkylcarbonyl group”. Specific examples of the “C₃₋₁₀cycloalkylcarbonyl group” include, for example, a cyclopropylcarbonyl group, a cyclobutylcarbonyl group, a cyclopentyl carbonyl group, a cyclohexyl carbonyl group, a cycloheptyl carbonyl group, a cyclooctyl carbonyl group, a cyclononyl carbonyl group, a cyclodecyl carbonyl group, an adamantyl carbonyl group, and the like.

A “C₁₋₆alkylsulfonyl group” refers to a sulfonyl group substituted with the above-described “C₁₋₆alkyl”, and is preferably a “C₁₋₄alkylsulfonyl group”. Specific examples of the “C₁₋₆alkylsulfonyl group” include, for example, a methylsulfonyl group, an ethylsulfonyl group, a propylsulfonyl group, an isopropylsulfonyl group, a butylsulfonyl group, a pentylsulfonyl group, a hexylsulfonyl group, and the like.

A “C₃₋₁₀cycloalkylsulfonyl group” refers to a sulfonyl group substituted with the above-described “C₃₋₁₀cycloalkyl”, and is preferably a “C₃₋₇cycloalkylsulfonyl group”. Specific examples of the “C₃₋₁₀cycloalkylsulfonyl group” include, for example, a cyclopropylsulfonyl group, a cyclobutylsulfonyl group, a cyclopentylsulfonyl group, a cyclohexylsulfonyl group, a cycloheptylsulfonyl group, and the like.

A “C₁₋₆alkyloxycarbonyl group” refers to a carbonyl group substituted with the above-described “C₁₋₆alkyloxy group”. Specific examples of the “C₁₋₆alkyloxycarbonyl group” include, for example, a methoxycarbonyl group, an ethoxycarbonyl group, and the like. In the relevant field, an “alkyloxycarbonyl group” is sometimes referred to as an “alkoxycarbonyl group”, however, both are synonymous.

A “C₃₋₁₀ cycloalkyloxycarbonyl group” refers to an oxycarbonyl group substituted with the above-described “C₃₋₁₀cycloalkyloxy group”, and is preferably a “C₃₋₇cycloalkyloxycarbonyl group”. Specific examples of the “C₃₋₁₀cycloalkyloxycarbonyl group” include, for example, a cyclopropyloxycarbonyl group, a cyclobutyloxycarbonyl group, a cyclopentyloxycarbonyl group, a cyclohexyloxycarbonyl group, a cycloheptyloxycarbonyl group, and the like.

A “C₆₋₁₀aryl group” refers to an aromatic hydrocarbon cyclic group of which the carbon number is six to ten. Specific examples of the “C₆₋₁₀aryl group” include, for example, a phenyl group, a 1-naphthyl group, a 2-naphthyl group, and the like. Particularly preferably, it includes a phenyl group. The “C₆₋₁₀aryl group” also encompasses a 8 to 14-membered polycyclic group in which an aromatic hydrocarbon ring and a C₄₋₆cycloalkyl ring are ring-fused, or a 9 to 14-membered polycyclic group in which an aromatic hydrocarbon ring is ring-fused to, for example, a 5 to 6-membered heterocycle having one to three homogeneous or heterogeneous atoms independently selected from the group consisting of a nitrogen atom, an oxygen atom, and a sulfur atom. Specific examples thereof include, for example, groups represented by the following:

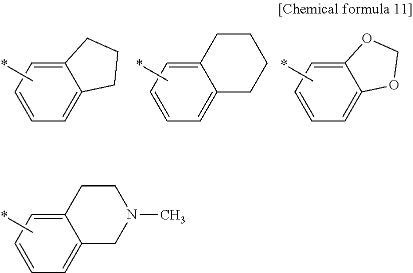

(wherein * is the point of attachment in the above)
and the like.

As used herein, an aromatic hydrocarbon ring refers to the ring portion of the above-described “C₆₋₁₀aryl group”.

A “5 to 12-membered monocyclic or polycyclic heteroaryl group” refers to a 5 to 7-membered monocyclic aromatic heterocyclic group or 8 to 12-membered bicyclic aromatic heterocyclic group that contains one to four atoms independently selected from the group consisting of a nitrogen atom, an oxygen atom, and a sulfur atom. It is preferably a “5 to 7-membered monocyclic heteroaryl group”.

Specific examples of the “5 to 12-membered monocyclic or polycyclic heteroaryl group” include a pyridyl group, a pyridazinyl group, an isothiazolyl group, a pyrrolyl group, a furyl group, a thienyl group, a thiazolyl group, an imidazolyl group, a pyrimidinyl group, a thiadiazolyl group, a pyrazolyl group, an oxazolyl group, an isoxazolyl group, a pyrazinyl group, a triazinyl group, a triazolyl group, an oxadiazolyl group, a triazolyl group, a tetrazolyl group, an indolyl group, an indazolyl group, a chromenyl group, a quinolyl group, an isoquinolyl group, a benzofuranyl group, a benzothienyl group, a benzoxazolyl group, a benzothiazolyl group, a benzisoxazolyl group, a benzisothiazolyl group, a benzotriazolyl group, a benzimidazolyl group, and the like. Examples of a preferable heteroaryl group include a pyridyl group, a pyrimidinyl group, a quinolyl group, and an isoquinolyl group. Further preferably, it includes a pyridyl group.

As used herein, an “aromatic heterocycle” refers to the ring portion of the above-described “5 to 12-membered monocyclic or polycyclic heteroaryl group”.

A “heterocyclic group” refers to a 3 to 10-membered heterocyclic group having one to three homogeneous or heterogeneous atoms independently selected from the group consisting of a nitrogen atom, an oxygen atom, and a sulfur atom, is preferably a 4 to 7-membered group, and is more preferably a 5 to 6-membered group. All of the nitrogen atoms, oxygen atoms, and sulfur atoms are atoms constituting the ring. The heterocyclic group may be any of a saturated, partially unsaturated, or unsaturated heterocyclic group, and a saturated heterocyclic group is further preferable. Specific examples of the “heterocyclic group” include an epoxy group, an aziridine group, an azetidine group, a pyranyl group, a tetrahydrofuryl group (a tetrahydrofuranyl group), a pyrrolidinyl group, an imidazolidinyl group, a piperidinyl group, a morpholinyl group, a thiomorpholinyl group, a dioxothiomorpholinyl group, a hexamethyleneiminyl group, an oxazolidinyl group, a thiazolidinyl group, an oxoimidazolidinyl group, a dioxoimidazolidinyl group, an oxooxazolidinyl group, a dioxooxazolidinyl group, a dioxothiazolidinyl group, a tetrahydropyridinyl group, an oxetanyl group, a tetrahydropyranyl group, and the like. It should be noted that the group also encompasses a heterocyclic group having a bridged structure. Regarding the group, a nitrogen atom constituting the ring cannot be at a position to be attached in “the group”. That is, the group does not encompass ideas of, for example, a 1-pyrrolidino group and the like.

A “3 to 10-membered saturated heterocyclic group” is preferably a saturated heterocyclic group that is composed of three to ten atoms including, other than a carbon atom, one or two atoms independently selected from the group consisting of a nitrogen atom, an oxygen atom, and a sulfur atom. It is more preferably a 4 to 7-membered monocyclic saturated heterocyclic group, and further preferably includes a 5 or 6-membered monocyclic saturated heterocyclic group, and the like. Specific examples of the “3 to 10-membered saturated heterocyclic group” include, for example, a tetrahydrofuryl group, a pyrrolidinyl group, an imidazolidinyl group, a piperidinyl group, a morpholinyl group, a thiomorpholinyl group, a dioxothiomorpholinyl group, a hexamethyleneiminyl group, an oxazolidinyl group, a thiazolidinyl group, an oxoimidazolidinyl group, a dioxoimidazolidinyl group, an oxooxazolidinyl group, a dioxooxazolidinyl group, a dioxothiazolidinyl group, a tetrahydrofuranyl group, an oxetanyl group, a tetrahydropyranyl group, and the like.

The above-described “heterocyclic group” may form a fused ring with a 6-membered aromatic hydrocarbon ring or a 6-membered aromatic heterocycle. Examples thereof include a bicyclic “heterocycle” having eleven or twelve ring-constituting atoms in which the above-described 5 or 6-membered “heterocyclic group” is fused to a 6-membered aromatic hydrocarbon ring or a 6-membered aromatic heterocycle. Examples of the 6-membered aromatic hydrocarbon ring include a benzene ring and the like. Examples of the 6-membered aromatic heterocycle include pyridine, pyrimidine, pyridazine, and the like. Specific examples of the fused-cyclic group include a dihydroindolyl group, a dihydroisoindolyl group, a dihydropurinyl group, a dihydrothiazolopyrimidinyl group, a dihydrobenzodioxanyl group, an isoindolyl group, an indazolyl group, a tetrahydroquinolinyl group, a tetrahydroisoquinolinyl group, a tetrahydronaphthyridinyl group, and the like.

A “3 to 10-membered saturated heterocyclyloxycarbonyl group” refers to an oxycarbonyl group substituted with the above-described “3 to 10-membered saturated heterocyclic group”, and is preferably an oxycarbonyl group substituted with a 4 to 7-membered saturated heterocyclic group, and is more preferably an oxycarbonyl group substituted with a 5 or 6-membered saturated heterocyclic group. Specific examples thereof include a tetrahydropyranyloxycarbonyl group and the like.

A “C₆₋₁₀aryloxy group” refers to an oxy group substituted with the above-described “C₆₋₁₀aryl group”, and is preferably a “C₆aryloxy group” (a phenoxy group). Specific examples of the “C₆₋₁₀aryloxy group” include, for example, a phenoxy group, a 1-naphthoxy group, a 2-naphthoxy group, and the like.

A “C₆₋₁₀arylcarbonyl group” refers to a carbonyl group substituted with the above-described “C₆₋₁₀aryl group”, and is preferably a “C₆arylcarbonyl group” (a phenylcarbonyl group). Specific examples of the “C₆₋₁₀arylcarbonyl group” include, for example, a benzoyl group, a 1-naphthoyl group, a 2-naphthoyl group, and the like.

A “5 to 12-membered monocyclic or polycyclic heteroarylcarbonyl group” refers to a carbonyl group substituted with the above-described “5 to 12-membered monocyclic or polycyclic heteroaryl group”, and is preferably a “5 to 7-membered monocyclic heteroarylcarbonyl group”. Specific examples of the “5 to 12-membered monocyclic or polycyclic heteroarylcarbonyl group” include, for example, a pyridylcarbonyl group and the like.

A “3 to 10-membered saturated heterocyclylcarbonyl group” refers to a carbonyl group substituted with the above-described “3 to 10-membered saturated heterocyclic group”. Specific examples of the “3 to 10-membered saturated heterocyclylcarbonyl group” include, for example, a tetrahydropyranylcarbonyl group and the like.

A “C₆₋₁₀arylsulfonyl group” refers to a sulfonyl group substituted with the above-described “C₆₋₁₀aryl group”, and is preferably a “C₆arylsulfonyl group”. Specific examples of the “C₆₋₁₀arylsulfonyl group” include, for example, a phenylsulfonyl group, a 1-naphthylsulfonyl group, a 2-naphthylsulfonyl group, and the like.

A “C₆₋₁₀aryloxycarbonyl group” refers to a carbonyl group substituted with the above-described “C₆₋₁₀aryloxy group”, and is preferably a “C₆aryloxycarbonyl group” (a phenyloxycarbonyl group). Specific examples of the “C₆₋₁₀aryloxycarbonyl group” include, for example, a phenoxycarbonyl group, 1-naphthoxycarbonyl group, 2-naphthoxycarbonyl group, and the like.

A “5 to 12-membered monocyclic or polycyclic heteroaryloxy group” refers to an oxy group substituted with the above-described “5 to 12-membered monocyclic or polycyclic heteroaryl group”. A “5 to 12-membered monocyclic or polycyclic heteroaryloxycarbonyl group” refers to a carbonyl group substituted with a “5 to 12-membered monocyclic or polycyclic heteroaryloxy group”, and examples thereof preferably include a “5 to 7-membered monocyclic heteroaryloxycarbonyl group”. Specific examples of the “5 to 12-membered monocyclic or polycyclic heteroaryloxycarbonyl group” include, for example, a pyridyloxycarbonyl group and the like.

An “optionally substituted amino group” refers to a “mono- or di-substituted amino group” or a “3 to 10-membered cyclic amino group”.

Examples of a substituent in the “mono- or di-substituted amino group” include, for example, a “C₁₋₆alkyl group”, a “C₃₋₁₀cycloalkyl group”, a “C₃₋₁₀cycloalkylC₁₋₄alkyl group”, a “C₃₋₇cycloalkylC₁₋₄alkyloxycarbonyl group”, a “C₁₋₄alkylcarbonyl group”, a “C₁₋₄alkyloxycarbonyl group”, a “3 to 10-membered saturated heterocyclic group”, a “3 to 10-membered saturated heterocyclylC₁₋₄alkyl group”, a “3 to 10-membered saturated heterocyclylcarbonyl group”, a “3 to 10-membered saturated heterocyclyloxycarbonyl group”, a “3 to 10-membered saturated heterocyclylC₁₋₄alkylcarbonyl group”, a “C₆₋₁₀aryl group”, a “C₆₋₁₀arylcarbonyl group”, a “C₆₋₁₀aryloxycarbonyl group”, a “5 or 6-membered monocyclic heteroaryl group”, a “5 or 6-membered monocyclic heteroarylC₁₋₄alkyl group”, and the like.

Specific examples of the “mono-substituted amino group” include, for example,

a “mono-C₁₋₆alkylamino group” (e.g., a methylamino group, an ethylamino group, a propylamino group, a 1-methylethylamino group, a butylamino group, a 2-methylpropylamino group, a 1-methylpropylamino group, a 1,1-dimethylethylamino group, and the like);
a “C₃₋₈cycloalkylamino group” (e.g., a cyclopropylamino group, a cyclobutylamino group, a cyclopentylamino group, a cyclohexylamino group, a cycloheptylamino group, and the like);
a “(C₃₋₈cycloalkylC₁₋₄alkyl)amino group” (e.g., a cyclopropylmethylamino group, a cyclobutylmethylamino group, a cyclopentylmethylamino group, a cyclohexylmethylamino group, a cycloheptylmethylamino group, and the like);
a “(C₃₋₈cycloalkyloxycarbonyl)amino group” (e.g., a cyclopropoxycarbonylamino group, a cyclobutoxycarbonylamino group, a cyclopentoxycarbonylamino group, a cyclohexyloxycarbonylamino group, a cycloheptyloxycarbonylamino group, and the like);
a “(C₁₋₄alkylcarbonyl)amino group” (e.g., a methylcarbonylamino group, an ethylcarbonylamino group, a propylcarbonylamino group, a 1-methylpropylcarbonylamino group, a 2-methylpropylcarbonylamino group, a butylcarbonylamino group, a 2,2-dimethylethylcarbonylamino group, and the like);
a “(C₁₋₄alkyloxycarbonyl)amino group” (e.g., a methoxycarbonylamino group, an ethoxycarbonylamino group, a propoxycarbonylamino group, a 1-methylpropoxycarbonylamino group, a 2-methylpropoxycarbonylamino group, a butoxycarbonylamino group, a 2,2-dimethylethoxycarbonylamino group, and the like);
a “C₆₋₁₀arylamino group” (e.g., a phenylamino group, a 1-naphthylamino group, a 2-naphthylamino group, and the like);
a “C₆₋₁₀arylcarbonylamino group” (e.g., a phenylcarbonylamino group, a 1-naphthylcarbonylamino group, a 2-naphthylcarbonylamino group, and the like);
a “C₆₋₁₀aryloxycarbonylamino group” (e.g., a phenoxycarbonylamino group, a 1-naphthoxycarbonylamino group, a 2-naphthoxycarbonylamino group, and the like);
a “3 to 10-membered saturated heterocyclylamino group” (e.g., a tetrahydropyranylamino group, a pyrrolidinylamino group, an oxopyrrolidinylamino group, a tetrahydrofuranylamino group, a piperidinylamino group, and the like);
a “(3 to 10-membered saturated heterocyclylC₁₋₄alkyl)amino group” (e.g., a tetrahydropyranylmethylamino group, a pyrrolidinylmethylamino group, an oxopyrrolidinylmethylamino group, a tetrahydrofuranylmethylamino group, a piperidinylmethylamino group, a piperazinylmethylamino group, a morpholinylmethylamino group, and the like);
a “3 to 10-membered saturated heterocyclylcarbonylamino group” (e.g., a tetrahydropyranylcarbonylamino group, a pyrrolidinylcarbonylamino group, an oxopyrrolidinylcarbonylamino group, a tetrahydrofuranylcarbonylamino group, a piperidinylcarbonylamino group, and the like);
a “3 to 10-membered saturated heterocyclyloxycarbonylamino group” (e.g., a tetrahydropyranyloxycarbonylamino group, a pyrrolidinyloxycarbonylamino group, an oxopyrrolidinyloxycarbonylamino group, a tetrahydrofuranyloxycarbonylamino group, a piperidinyloxycarbonylamino group, and the like);
a “(5 or 6-membered monocyclic heteroaryl)amino group” (e.g., a pyrrolylamino group, a thienylamino group, a furylamino group, an oxazolylamino group, a thiazolylamino group, an isoxazolylamino group, an isothiazolylamino group, an imidazolylamino group, a pyrazolylamino group, a triazolylamino group, an oxadiazolylamino group, a thiadiazolylamino group, a tetrazolylamino group, a pyridylamino group, a pyrazylamino group, a pyrimidylamino group, a pyridazylamino group, a triazylamino group, and the like);
a “(5 or 6-membered monocyclic heteroarylC₁₋₄alkyl)amino group” (e.g., a pyrrolylmethylamino group, a thienylmethylamino group, a furylmethylamino group, an oxazolylmethylamino group, a thiazolylmethylamino group, an isoxazolylmethylamino group, an isothiazolylmethylamino group, an imidazolylmethylamino group, a pyrazolylmethylamino group, a triazolylmethylamino group, an oxadiazolylmethylamino group, a thiadiazolylmethyl amino group, a tetrazolylmethylamino group, a pyridylmethylamino group, a pyrazylmethylamino group, a pyrimidylmethylamino group, a pyridazylmethylamino group, a triazylmethylamino group, and the like); and the like.

Specific examples of the “di-substituted amino group” include, for example,

a “di-C₁₋₆alkylamino group” (e.g., a dimethylamino group, a diethylamino group, a dipropylamino group, a di(1-methylethyl)amino group, a dibutylamino group, a di(2-methylpropyl)amino group, a di(1-methylpropyl)amino group, a di(1,1-dimethylethyl)amino group, and the like);
a “N—(C₁₋₆alkyl)-N—(C₃₋₁₀cycloalkyl)amino group” (e.g., a methylcyclopropylamino group, a methylcyclobutylamino group, a methylcyclopentylamino group, a methylcyclohexylamino group, a methylcycloheptylamino group, and the like);
a “N—(C₁₋₆alkyl)-N-(4 to 7-membered saturated heterocyclyl)amino group” (e.g., a methyltetrahydropyranylamino group, a methylpyrrolidinylamino group, a methyloxopyrrolidinylamino group, a methyltetrahydrofuranylamino group, a methylpiperidinylamino group, and the like); and the like.

A “3 to 10-membered cyclic amino group” refers to a 3 to 10-membered monocyclic cyclic amino group having one to three homogeneous or heterogeneous heteroatoms selected from the group consisting of a nitrogen atom, an oxygen atom, and a sulfur atom, and is preferably a 5 or 6-membered monocyclic cyclic amino group. Regarding the “3 to 10-membered cyclic amino group”, a nitrogen atom constituting the ring is at a position to be attached in the “group”. Specific examples of the “3 to 10-membered cyclic amino” include, for example, an azetidino group, a pyrrolidino group, an imidazolidino group, an oxazolidino group, a thiazolidino group, a piperazino group, a piperidino group, a morpholino group, a thiomorpholino group, an azepano group, an oxoazepano group, and the like. It should be noted that the group also encompasses a cyclic amino group of which the ring contains a partially unsaturated bond.

A “3 to 10-membered cyclic amino group” may form a fused ring with a 3 to 6-membered cycloalkyl ring, a 6-membered aromatic hydrocarbon ring, or a 5 or 6-membered aromatic heterocycle. Specific examples of a cyclic amino group forming such a fused ring include “groups” represented by the following:

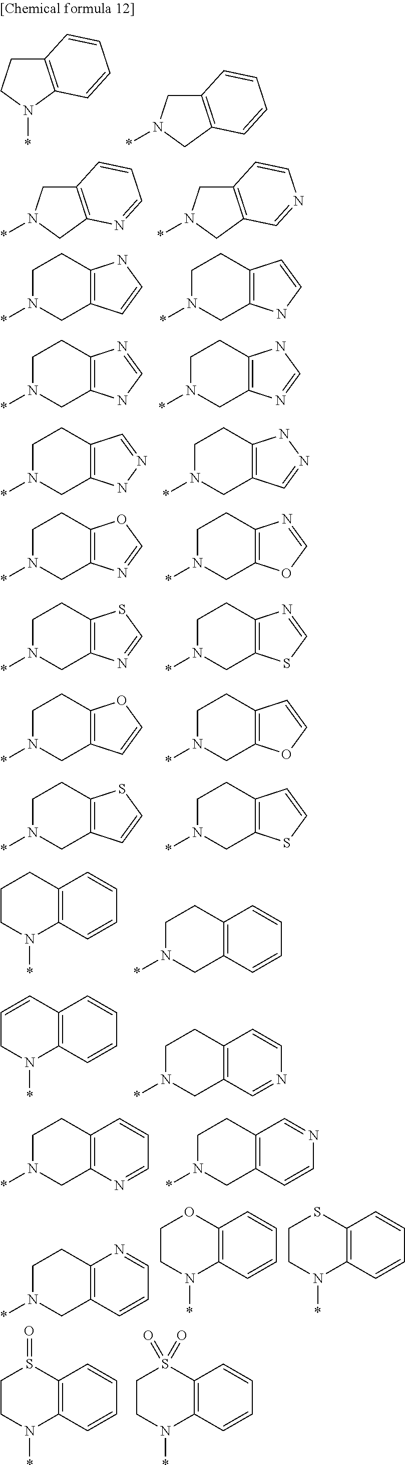

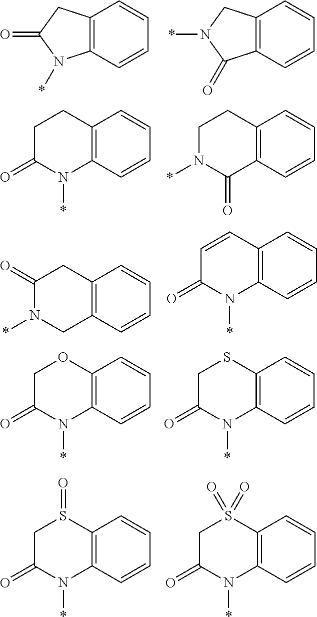

(wherein * is the point of attachment in the above)
and the like.

An “aminocarbonyl group” refers to a carbonyl group substituted with the above-described “optionally substituted amino group”.

An “aminosulfonyl group” refers to a sulfonyl group substituted with the above-described “optionally substituted amino group”.

A “C₁₋₆alkylaminocarbonyl group” refers to a group that is the above-described “mono- or di-substituted amino group” in which the amino group substituted with one or two C₁₋₆alkyl groups is bound to a carbonyl group.

A “C₃₋₁₀cycloalkylaminocarbonyl group” refers to a group that is the above-described “mono- or di-substituted amino group” in which the amino group substituted with one or two C₃₋₁₀cycloalkyl groups is bound to a carbonyl group.

A “C₆₋₁₀arylaminocarbonyl group” refers to a group that is the above-described “mono- or di-substituted amino group” in which the amino group substituted with one or two C₆₋₁₀aryl groups is bound to a carbonyl group.

A “5 to 12-membered monocyclic or polycyclic heteroarylaminocarbonyl group” refers to a group that is the above-described “mono- or di-substituted amino group” in which the amino group substituted with one or two 5 to 12-membered monocyclic or polycyclic heteroaryl groups is bound to a carbonyl group.

A “3 to 10-membered saturated heterocyclylaminocarbonyl group” refers to a group that is the above-described “mono- or di-substituted amino group” in which the amino group substituted with one or two 3 to 10-membered saturated heterocycles is bound to a carbonyl group.

Examples of a substituent of an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, an optionally substituted 5 to 12-membered monocyclic or polycyclic heteroaryl group, an optionally substituted C₁₋₆alkylsulfonyl group, an optionally substituted C₃₋₁₀cycloalkylsulfonyl group, an optionally substituted C₆₋₁₀arylsulfonyl group, an optionally substituted aminosulfonyl group, an optionally substituted 3 to 10-membered cyclic aminosulfonyl group, an optionally substituted C₁₋₆alkylcarbonyl group, an optionally substituted C₃₋₁₀cycloalkylcarbonyl group, an optionally substituted C₆₋₁₀arylcarbonyl group, an optionally substituted 3 to 10-membered saturated heterocyclylcarbonyl group, an optionally substituted 5 to 12-membered monocyclic or polycyclic heteroarylcarbonyl group, an optionally substituted C₁₋₆alkyloxycarbonyl group, an optionally substituted C₃₋₁₀cycloalkyloxycarbonyl group, an optionally substituted C₆₋₁₀aryloxycarbonyl group, an optionally substituted 3 to 10-membered saturated heterocyclyloxycarbonyl group, an optionally substituted 5 to 12-membered monocyclic or polycyclic heteroaryloxycarbonyl group, an optionally substituted C₁₋₆alkylaminocarbonyl group, an optionally substituted C₃₋₁₀cycloalkylaminocarbonyl group, an optionally substituted C₆₋₁₀arylaminocarbonyl group, an optionally substituted 3 to 10-membered saturated heterocyclylaminocarbonyl group, and an optionally substituted 5 to 12-membered monocyclic or polycyclic heteroarylaminocarbonyl group include, for example, substituents each of which is independently selected from the group consisting of

(1) a halogen atom,
(2) a hydroxyl group,
(3) a carboxyl group,
(4) a sulfinate group,
(5) a sulfonate group,
(6) a phosphonate group,
(7) a C₁₋₆alkyl group,
(8) a C₃₋₁₀cycloalkyl group,
(9) a C₆₋₁₀aryl group,
(10) a C₅₋₁₀heteroaryl group,
(11) a C₁₋₆alkyloxy group,
(12) a C₃₋₁₀cycloalkyloxy group,
(13) a C₁₋₆alkyloxycarbonyl group,
(14) a C₁₋₆alkylcarbonyl group,
(15) a 3 to 10-membered saturated heterocyclic group,
(16) a 5 to 12-membered monocyclic or polycyclic heteroaryl group,
(17) —NR⁵R⁶,
(18) —CO₂R⁵,
(19) a guanidine group,
(20) —CONR⁵R⁶,
(21) —SO₂R⁵,
(22) —SO₂NR⁵R⁶,
(23) a cyano group,
(24) —OCO₂R⁵,
(25) —OCONR⁵R⁶, and
(26) —NR₅CO₂R⁶.
Preferably, examples thereof include substituents each of which is independently selected from the group consisting of (a) a halogen atom, (b) a hydroxyl group, (c) a C₁₋₆alkyl group, or (d) a C₁₋₆alkyloxy group.

R⁵ and R⁶ are each independently a hydrogen atom or a C₁₋₆alkyl group; or R⁵ and R⁶, when attached to a nitrogen atom, may be taken together with the nitrogen atom to form a 3 to 10-membered cyclic amino group.

Preferable embodiments of the present invention are further described.

In the present specification, formula (I) is an isomer having a relationship with the following formula (I′) between an oxidant and a reductant that can attain equilibrium. They can be considered as being synonymous.

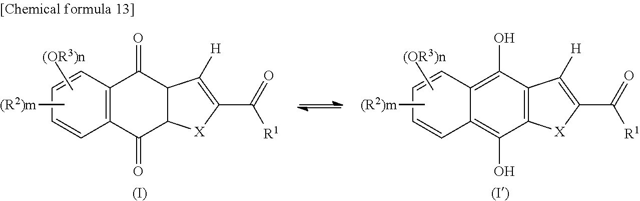

In formula (I), X is an oxygen atom or a sulfur atom, and preferably an oxygen atom; R¹ is a hydrogen atom, a cyano group, a trifluoromethyl group, a trifluoromethoxy group, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, an optionally substituted 5 to 12-membered monocyclic or polycyclic heteroaryl group, —OR⁴, or —SR⁴,

preferably a C₁₋₆alkyl group optionally substituted with one to three halogen atoms, hydroxyl groups, one or two C₁₋₆alkyloxy groups, or a 3 to 10-membered saturated heterocyclic group, and further preferably a methyl group;
each R² is independently a halogen atom, a cyano group, a nitro group, a trifluoromethyl group, a trifluoromethoxy group, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted C₁₋₆alkylcarbonyl group, an optionally substituted C₃₋₁₀cycloalkylcarbonyl group, an optionally substituted C₆₋₁₀arylcarbonyl group, an optionally substituted 5 to 12-membered monocyclic or polycyclic heteroarylcarbonyl group, an optionally substituted 3 to 10-membered saturated heterocyclic group, an optionally substituted C₆₋₁₀aryl group, an optionally substituted 5 to 12-membered monocyclic or polycyclic heteroaryl group, an optionally substituted C₁₋₆alkylsulfonyl group, an optionally substituted C₃₋₁₀cycloalkylsulfonyl group, an optionally substituted C₆₋₁₀arylsulfonyl group, an optionally substituted aminosulfonyl group, —N(R⁴)₂, or —SR⁴,
preferably, each independently, a halogen atom, a cyano group, an optionally substituted C₁₋₆alkyl group, an optionally substituted C₂₋₆alkenyl group, an optionally substituted C₂₋₆alkynyl group, an optionally substituted C₃₋₁₀cycloalkenyl group, an optionally substituted C₁₋₆alkylcarbonyl group, an optionally substituted C₆₋₁₀aryl group, or an optionally substituted 5 to 12-membered monocyclic or polycyclic heteroaryl group,
further preferably, each independently, a halogen atom, an optionally substituted C₁₋₆alkyl group, an optionally substituted C₆₋₁₀aryl group, or an optionally substituted 5 to 12-membered monocyclic or polycyclic heteroaryl group, and
particularly preferably, each independently, a halogen atom;
each R³ is independently a hydrogen atom, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, an optionally substituted 5 to 12-membered monocyclic or polycyclic heteroaryl group, an optionally substituted C₁₋₆alkylcarbonyl group, an optionally substituted C₃₋₁₀cycloalkylcarbonyl group, an optionally substituted C₆₋₁₀arylcarbonyl group, an optionally substituted 3 to 10-membered saturated heterocyclylcarbonyl group, an optionally substituted 5 to 12-membered monocyclic or polycyclic heteroarylcarbonyl group, an optionally substituted C₁₋₆alkyloxycarbonyl group, an optionally substituted C₃₋₁₀cycloalkyloxycarbonyl group, an optionally substituted C₆₋₁₀aryloxycarbonyl group, an optionally substituted 3 to 10-membered saturated heterocyclyloxycarbonyl group, an optionally substituted 5 to 12-membered monocyclic or polycyclic heteroaryloxycarbonyl group, an optionally substituted C₁₋₆alkylaminocarbonyl group, an optionally substituted C₃₋₁₀cycloalkylaminocarbonyl group, an optionally substituted C₆₋₁₀arylaminocarbonyl group, an optionally substituted 3 to 10-membered saturated heterocyclylaminocarbonyl group, or an optionally substituted 5 to 12-membered monocyclic or polycyclic heteroarylaminocarbonyl group,
preferably, each independently, a hydrogen atom, an optionally substituted C₁₋₆alkyl group, an optionally substituted C₆₋₁₀aryl group, an optionally substituted 5 to 12-membered monocyclic or polycyclic heteroaryl group, an optionally substituted C₁₋₆alkylcarbonyl group, an optionally substituted C₆₋₁₀arylcarbonyl group, an optionally substituted C₁₋₆alkyloxycarbonyl group, an optionally substituted C₁₋₆aryloxycarbonyl group, an optionally substituted C₁₋₆alkylaminocarbonyl group, or an optionally substituted arylaminocarbonyl group, and further preferably, each independently, a hydrogen atom, an optionally substituted C₁₋₆alkyl group, an optionally substituted C₆₋₁₀arylcarbonyl group, or an optionally substituted C₁₋₆alkyloxycarbonyl group;
each R⁴ is independently a hydrogen atom, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, or an optionally substituted 5 to 12-membered monocyclic or polycyclic heteroaryl group, or when two R⁴ are attached to a nitrogen atom, the two R⁴ may be taken together with the nitrogen atom to which they are attached to form a nitrogen-containing 3 to 10-membered saturated heterocyclic group,
preferably, each independently, a hydrogen atom, an optionally substituted C₁₋₆alkyl group, an optionally substituted C₃₋₁₀cycloalkyl group, an optionally substituted C₆₋₁₀aryl group, or an optionally substituted 5 to 12-membered monocyclic or polycyclic heteroaryl group, or when two R⁴ are attached to a nitrogen atom, the two R⁴ may be taken together with the nitrogen atom to which they are attached to form a nitrogen-containing 3 to 10-membered saturated heterocyclic group,
further preferably, each independently, a hydrogen atom or an optionally substituted C₁₋₆alkyl group, or when two R⁴ are attached to a nitrogen atom, the two R⁴ may be taken together with the nitrogen atom to which they are attached to form a nitrogen-containing 3 to 10-membered saturated heterocyclic group; and
m is 0, 1, 2, 3, or 4, and n is 1, 2, 3, or 4 (with the proviso that the sum of m and n is 1, 2, 3, or 4), and
preferably m and n are both 1.

Examples of preferable compounds in the present invention include compounds selected from the group consisting of the following compounds, or pharmaceutically acceptable salts thereof:
2-acetoxy-8-chloro-5-hydroxynaphtho[2,3-b]furan-4,9-dione;
2-acetyl-6-bromo-5-hydroxynaphtho[2,3-b]furan-4,9-dione;
2-acetyl-7-bromo-8-hydroxynaphtho[2,3-b]furan-4,9-dione;
2-acetyl-6-butyl-5-hydroxynaphtho[2,3-b]furan-4,9-dione;
2-acetyl-5-hydroxy-6-(4-methoxyphenyl)naphtho[2,3-b]furan-4,9-dione;
2-acetyl-5-hydroxy-(2-methoxypyrimidin-5-yl)naphtho[2,3-b]furan-4,9-dione;
2-acetyl-6-bromo-5-(methoxymethoxy)naphtho[2,3-b]furan-4,9-dione;
2-acetyl-6-bromo-4,9-dioxo-4,9-dihydronaphtho[2,3-b]furan-5-yl benzoate;
2-acetyl-6-bromo-4,9-dioxo-4,9-dihydronaphtho[2,3-b]furan-5-yl 2-methylpropyl carbonate;
2-acetyl-6-chloro-5-hydroxynaphtho[2,3-b]furan-4,9-dione;
2-acetyl-7-chloro-5-hydroxynaphtho[2,3-b]furan-4,9-dione;
2-acetyl-5-chloro-8-hydroxynaphtho[2,3-b]furan-4,9-dione;
2-acetyl-6-chloro-8-hydroxynaphtho[2,3-b]furan-4,9-dione;
2-acetyl-7-chloro-8-hydroxynaphtho[2,3-b]furan-4,9-dione;
2-acetyl-8-chloro-7-hydroxynaphtho[2,3-b]furan-4,9-dione;
2-acetyl-6-chloro-7-hydroxynaphtho[2,3-b]furan-4,9-dione;
2-acetyl-5-chloro-7-hydroxynaphtho[2,3-b]furan-4,9-dione;
2-acetyl-5-chloro-6-hydroxynaphtho[2,3-b]furan-4,9-dione;
2-acetyl-7-chloro-6-hydroxynaphtho[2,3-b]furan-4,9-dione; and
2-acetyl-8-chloro-6-hydroxynaphtho[2,3-b]furan-4,9-dione.

Examples of further preferable compounds include compounds selected from the group consisting of the following compounds, or pharmaceutically acceptable salts thereof:
2-acetoxy-8-chloro-5-hydroxynaphtho[2,3-b]furan-4,9-dione;
2-acetyl-6-bromo-5-hydroxynaphtho[2,3-b]furan-4,9-dione; and
2-acetyl-7-bromo-8-hydroxynaphtho[2,3-b]furan-4,9-dione.

Examples of a “pharmaceutically acceptable salt” include acid addition salts and base addition salts of compounds of formula (I). Examples of the acid addition salts include: inorganic acid salts such as hydrochloride, hydrobromide, sulfate, hydroiodide, nitrate, phosphate, and the like; and organic acid salts such as citrate, oxalate, phthalate, fumarate, maleate, succinate, malate, acetate, formate, propionate, benzoate, trifluoroacetate, methanesulfonate, benzenesulfonate, p-toluenesulfonate, camphorsulfonate, and the like. Examples of the base addition salts include: inorganic base salts such as sodium, potassium, calcium, magnesium, barium, aluminum salts, and the like; and organic base salts such as trimethylamine, triethylamine, pyridine, picoline, 2,6-lutidine, ethanolamine, diethanolamine, triethanolamine, tromethamine [tris(hydroxymethyl)methylamine], tert-butylamine, cyclohexylamine, dicyclohexylamine, N,N-dibenzylethylamine, and the like. Further examples include salts of an amino acid such as basic and acidic amino acids including arginine, lysine, ornithine, aspartic acid, glutamic acid, and the like.

Suitable salts and pharmaceutically acceptable salts of starting compounds, intermediate compounds, and target compounds are conventional nontoxic salts. Examples thereof include acid addition salts such as organic acid salts (e.g., acetate, trifluoroacetate, maleate, fumarate, citrate, tartrate, methanesulfonate, benzenesulfonate, formate, p-toluenesulfonate, or the like) and inorganic acid salts (e.g., hydrochloride, hydrobromide, hydroiodide, sulfate, nitrate, phosphate, or the like), salts with an amino acid (e.g., arginine, aspartic acid, glutamic acid, or the like), metal salts such as alkali metal salts (e.g., sodium salt, potassium salt, or the like), alkali earth metal salts (e.g., calcium salt, magnesium salt, or the like), and the like, ammonium salts, organic base salts (e.g., trimethylamine salts, triethylamine salts, pyridine salts, picoline salts, dicyclohexylamine salts, N,N′-dibenzylethylene diamine salts, or the like), and the like, and additionally those skilled in the art can appropriately select them.

When it is desired to obtain a salt of the present compound, if the present compound is obtained in a salt form, it may be purified as it is, or if it is obtained in free form, it may be dissolved or suspended in a suitable organic solvent, followed by addition of an acid or a base to form a salt in accordance with a general method.

In addition, although the present compounds and pharmaceutically acceptable salts thereof may be present in an adduct form with water or any kind of solvent, these adducts are also encompassed by the present invention.

In addition, the present invention encompasses compounds represented by formula (I) or prodrugs thereof, or pharmaceutically acceptable salts thereof. Further, the present compounds may be present in the form of a hydrate and/or a solvate, and these hydrates or solvates (such as ethanol solvates and the like) are also encompassed by the present compounds. Moreover, the present invention encompasses all tautomers and all present stereoisomers of the present compound (I) as well as those in all modes of crystal forms. As used herein, unless otherwise specified, the term “solvate” refers to a compound of the present invention that further comprises an amount of a solvent in a stoichiometric or non-stoichiometric ratio which solvent is bound noncovalently by intermolecular force; or salts thereof. In the present invention, one or two or more types of the solvates can be used in combination. When the solvent is water, the solvate is a hydrate.

The term “a prodrug of a compound of formula (I)” in the present specification refers to a compound that is converted to a compound of formula (I) by reaction with an enzyme, gastric acid, or the like under physiological condition in vivo, for example, a compound that is converted to a compound of formula (I) by enzymatic oxidation, reduction, hydrolysis, or the like.

Among the present compounds (I), there are compounds that may be present as enantiomers based on an optically-active center, other stereoisomers, tautomers, geometric isomer, and the like. However, all possible isomers and mixtures thereof, racemates, and the like, including these, are encompassed within the scope of the present invention.

In particular, an enantiomer can be obtained as a racemic compound, or an optically-active substance when an optically-active starting material or intermediate is used, respectively. If necessary, in an appropriate step of the below-described production method, a corresponding starting material, intermediate, or racemic compound as a final product can be physically or chemically resolved into their optical enantiomers by a known separation method, such as a method using an optically active column, a fractional crystallization method, or the like. Specifically, for example, in a diastereomer method, two types of diastereomers are formed from a racemic compound by reaction using an optical active resolving agent. Since these different diastereomers generally have different physical properties, they can be separated by a known method such as fractional crystallization and the like.

In addition, a compound represented by the general formula (I) may be labeled by one or more isotopes (e.g., ²H(D), ³H, ¹⁴C, ³⁵S, and the like).

Production Method of the Present Compounds (I)

Hereinafter, production methods of compounds represented by formula (I) in the present invention are described with examples. However, the scope of present invention is certainly not limited to these examples.

The present compound represented by formula (I) or a salt thereof can be produced from a known compound, for example, the following production methods: Methods 1 to 3, and methods in accordance therewith, or by appropriately combining synthesis methods well-known to those skilled in the art.

It should be noted that a compound in a reaction includes the case where it forms a salt, and examples of such a salt include salts similar to salts in the compound (1), and the like.

In addition, a compound obtained in each step can be used in a next reaction as a reaction solution or as a crude product. However, it can be isolated from a reaction mixture in accordance with a routine method, and readily purified by a separation means such as recrystallization, distillation, chromatography, and the like.

With regard to raw material compounds, reaction reagents, and solvents, unless specifically stated otherwise, those commercially available were used or those produced in accordance with a known method were used.

Production Method of Compound (1-3)

### Production Method 1:

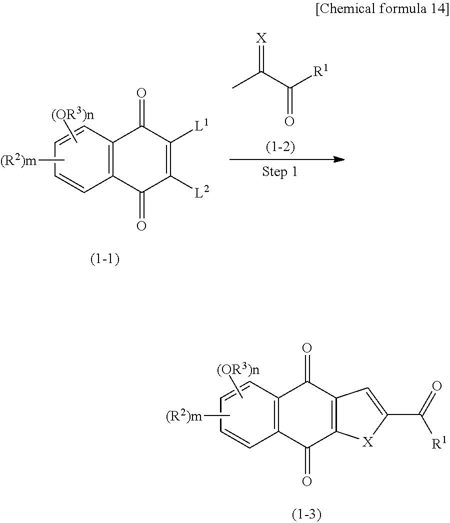

In the formulas, R¹, R², R³, m, n, and X are defined the same as described in the above item 1. L¹ and L² represent a hydrogen atom, a hydroxyl group, or a leaving group (e.g., chlorine atom, bromine atom, iodine atom, methanesulfonyloxy group, trifluoromethanesulfonyloxy group, p-toluenesulfonyloxy group, and the like).

Step 1

Compound (1-3) can be produced by reacting Compound (1-1) with Compound (1-2) in an organic solvent in the presence of a base. Examples of organic solvents used in the present reaction include aprotic solvent such as N,N-dimethylformamide, N-methyl-2-pyrrolidone, dimethyl sulfoxide, and the like; ether type solvent such as tetrahydrofuran, 1,4-dioxane, and the like; halogenated hydrocarbons such as dichloromethane, chloroform, 1,2-dichloroethane, chlorobenzene, and the like; hydrocarbons such as toluene, benzene, and the like; mixed solvents thereof; and the like; and suitably include N,N-dimethylformamide, N-methyl-2-pyrrolidone, dimethyl sulfoxide, acetonitrile, and tetrahydrofuran. In addition, it can be produced in a two-layer system of organic solvent-water. Both of organic bases and inorganic bases can be used as a base. Examples of the organic bases include 1-hydroxybenzotriazole, N-methylmorpholine, triethylamine, diisopropylethylamine, tributylamine, 1,8-diazabicyclo[5.4.0]undec-7-ene, 1,5-diazabicyclo[4.3.0]non-5-ene, 1,4-diazabicyclo[5.4.0]undec-7-ene, pyridine, dimethylaminopyridine, picoline, and the like. Examples of the inorganic bases include alkali halides such as potassium fluoride and the like; alkali hydroxide such as sodium hydroxide, potassium hydroxide, and the like; alkali carbonate such as sodium carbonate, potassium carbonate, cesium carbonate, sodium hydrogen carbonate, and the like; alkali alkoxide such as sodium ethoxide, sodium tert-butoxide, potassium tert-butoxide, and the like; alkali metal such as n-butyl lithium, methyl lithium, isopropylmagnesium bromide, and the like. The amount of compound (1-2) used is generally 1 to 5 mol, preferably 1.2 to 3 mol, per mol of compound (1-1). The amount of a base used is generally 2 to 10 mol, preferably 2 to 3 mol, per mol of compound (1-1). A reaction time is generally about 0.5 to about 48 hours, preferably about 0.5 to about 12 hours. A reaction temperature is generally about −20 to about 180° C., preferably about 0 to about 150° C.

With regard to Compound (1-1), a commercially available product thereof is used, or it can be produced according to a known method, for example, a method described in Journal of Medicinal Chemistry, 1329, vol. 29, (1986); European Journal of Medicinal Chemistry, 3938, vol. 45, (2010); Bioorganic & Medicinal Chemistry Letters, 952, vol. 21, (2011); European Journal of Organic Chemistry, 4201, vol. 18, (2006); Journal of Organic Chemistry, 5026, vol. 78, (2013); or the like.

With regard to Compound (1-2), a commercially available product thereof is used, or it can be produced according to a known synthesis method, for example, a method described in Bioorganic & Medicinal Chemistry, 5705, vol. 20, (2012); Journal of American Chemical Society, 3460, vol. 103, (1981); Journal of Medicinal Chemistry, 1347, vol. 40, (1997); Journal of Medicinal Chemistry, 5233, vol. 45, (2002); Organic Letters, 2856, vol. 11, (2009); or the like.

Production Method 2:

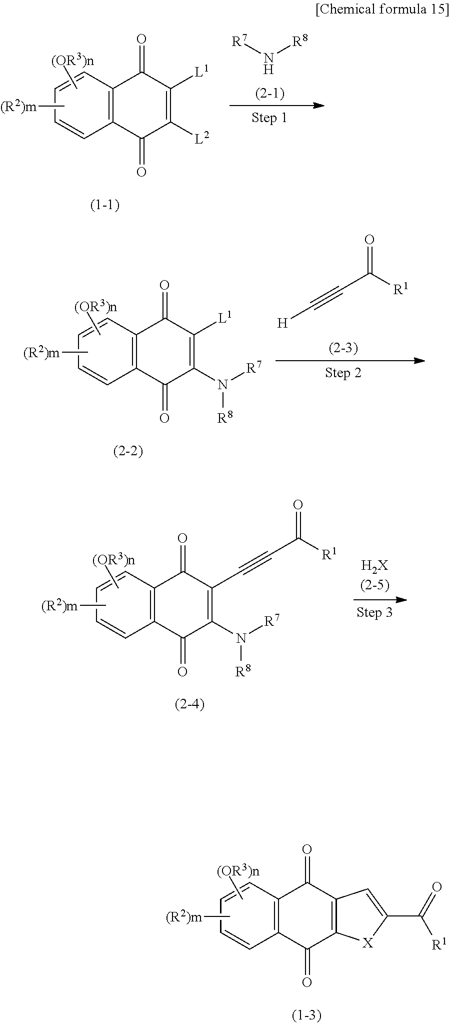

In the formulas, R¹, R², R³, X, m, and n are defined the same as described in the above item 1, L¹ and L² are defined the same as described in the above Production method 1, R⁷ and R⁸ are each independently an alkyl group (e.g., a methyl group, an ethyl group, a propyl group, and the like).

Compound (1-3) can be produced via Compound (2-2) and Compound (2-4) using Compound (1-1) as a raw material.

Step 1 is a step of reacting Compound (1-1) with Amine compound (2-1) to produce Compound (2-2) substituted with an amino group. A solvent to dissolve Compound (1-1) is not particularly limited as long as it is organic solvent, however, is preferably toluene. Preferable solvent for an amine is water, hexane, tetrahydrofuran, diethyl ether, toluene, or ethanol. The present step can be carried out at a temperature from −78° C. to the reflux temperature of a solvent, however, preferably in a range from −78 to −40° C.

Step 2 is a step of reacting Compound (2-2) (obtained in Step 1) with Acetylene compound (2-3) in an aprotic polar solvent in the presence of a copper catalyst and a base to produce Compound (2-4) substituted with acetylene. A preferable aprotic solvent in the present step is N,N-dimethylformamide or pyridine. When pyridine is used as a base in the present step, pyridine also functions as aprotic solvent. In the present step, one or more equivalents of Acetylene compound (2-3) are needed per equivalent of Compound (2-2), while 5 to 10 equivalents are preferable.

Step 3 is a step of cyclizing Compound (2-4) (obtained in Step 2) with Compound (2-5) to produce targeted Compound (1-3). The present step can be carried out in the presence of H₂X, in a H₂X/alcohol-type mixture, in a H₂X/acetonitrile mixture, or in a H₂X/acetone mixture. Examples of the alcohol-type solvent include methanol, ethanol, isopropanol, n-propanol, butanol, and the like. Preferable volume ratios of the H₂X/alcohol-type mixture, the H₂X/acetonitrile mixture, and the H₂X/acetone mixture are each 2/1. The reaction in the present step can be carried out at a temperature from 0° C. to the reflux temperature of a solvent, while the reflux temperature of a solvent is preferable.

The present steps 1 to 3 can be carried out according to a known method, for example, the same method as described in US 2012/0077986 A1 or the like.

Production Method 3:

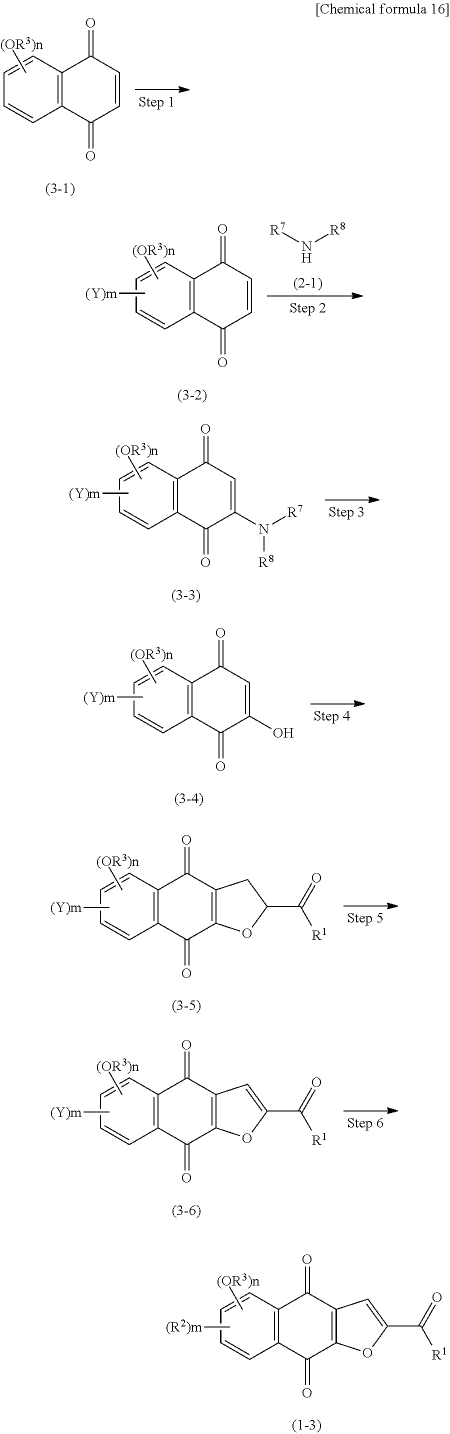

In the formulas, R¹, R², R³, m, and n are defined the same as described in the above item 1, R⁷ and R⁸ are defined the same as described in the above Production method 2, and Y represents a halogen atom (e.g., a chlorine atom, a bromine atom, an iodine atom).

Compound (1-3) can be produced via Compound (3-2) to Compound (3-6) using Compound (3-1) as a raw material.

The present Step 1 is a step of producing Compound (3-2) from Compound (3-1), and can be carried out according to a known method, for example, the same method as described in Liebigs Annalen der Chemie, 2420, vol. 12, (1985) or the like.

The present Steps 2 to 5 are steps of producing Compound (3-6) substituted with Y from Compound (3-2) obtained in Step 1, and can be carried out according to a known method, for example, the same method as described in Bioorganic & Medicinal Chemistry, 6286, vol. 17, (2009) or the like.

The present Step 6 is a step of carrying out a substitution reaction on the benzene ring of Compound (3-6) obtained in Step 5 to produce targeted Compound (1-3).

The present step can be carried out by reacting a boronic acid compound or organic tin compound, corresponding to R², with a palladium catalyst, a ligand, and a base in an inert solvent. Examples of the palladium catalyst used in the present step include palladium acetate, tetrakis(triphenylphosphine)palladium, tris(dibenzylideneacetone)dipalladium, and the like; examples of the ligand include triphenylphosphine, tri-o-tolylphosphine, tri-tert-butylphosphine, and the like; examples of the base include sodium carbonate, potassium carbonate, cesium carbonate, and the like; however, they are not particularly limited thereto. Examples of a preferable inert solvent in the present step include dimethoxyethane, 1,4-dioxane, toluene, ethanol, water, and mixture solvents thereof, and the like. The reaction of the present step can be carried out at a temperature from 0° C. to the reflux temperature of a solvent, while the reflux temperature of a solvent is preferable.

In respective reactions of the production methods described above, even in a case other than the case where the use of a protecting group is specifically and explicitly indicated, if any functional group other than a reaction point is modified under a described reaction condition or is unsuitable for performing the described method, a target compound can be obtained by protecting any point other than the reaction point as necessary, and deprotecting after the reaction is finished or a series of reactions are performed.

As a protecting group, conventional protecting groups can be used, such as known protecting groups, for example, those described in Protective Groups in Organic Synthesis, 3^{rd} ed., T. W. Greene, John Wiley & Sons Inc. (1999) or the like. Specifically, examples of a protecting group for an amino group include benzyloxycarbonyl, tert-butoxycarbonyl, acetyl, benzyl, and the like; and examples of a protecting group for a hydroxyl group include trialkylsilyl groups such as trimethylsilyl, tert-butyldimethylsilyl, and the like, acetyl, benzyl, and the like, respectively.

Introduction and removal of a protecting group can be carried out according to a method commonly used in synthetic organic chemistry (refer to, for example, the aforementioned Protective Groups in Organic Synthesis) or a method in accordance therewith.

In addition, intermediates or final products in the above-described production methods can be derived to other compounds encompassed by the present invention by appropriately converting a functional group thereof, and in particular, extending any kind of side chain using an amino group, a hydroxyl group, a carbonyl group, a halogen group, or the like as an aid, and at this time, as necessary, carrying out the above-described protection and deprotection. Conversion of a functional group and extension of a side chain can be carried out according to a routine, general method (refer to, for example, Comprehensive Organic Transformations, R. C. Larock, John Wiley & Sons Inc. (1999), and the like).

Intermediates and target compounds in the above-described respective production methods can be subjected to a routine purification method in synthetic organic chemistry, for example, neutralization, filtration, extraction, washing, drying, concentration, recrystallization, any kind of chromatography, and the like to perform isolation and purification. In addition, the intermediates can be subjected to a next reaction without particular purification.

Among raw materials and intermediates in respective production methods described above, those of which production methods are not particularly and repeatedly described are commercially available compounds or can be synthesized from a commercially available compound according to a known method to those skilled in the art or a method in accordance therewith.

The present compound is provided, for example, as an anticancer agent, and its target includes solid cancer as well as hematological cancer, such as lymphoma and leukemia. Although the type of cancer to which it is applied is not limited, specific examples thereof can include acute leukemia, chronic lymphocytic leukemia, chronic myelocytic leukemia, polycythemia vera, malignant lymphoma, brain tumor, head and neck cancer, esophageal cancer, thyroid cancer, small cell lung cancer, non-small-cell lung cancer, breast cancer, gastric cancer, gallbladder⋅bile duct cancer, hepatoma, pancreatic cancer, colon cancer, rectal cancer, chorioepithelioma, chorioblastoma, choriocarcinoma, endometrial cancer, cervical cancer, urothelial cancer, renal cell cancer, testicular tumor, Wilms' tumor, skin cancer, malignant melanoma, neuroblastoma, osteosarcoma, Ewing's sarcoma, and soft tissue sarcoma. An anticancer agent is an agent that is used for the purposes of preventing and/or treating cancer and has an effect to reduce or annihilate carcinoma or to inhibit the growth of carcinoma. Accordingly, an anticancer agent in the present invention also can be expressed as a (pharmaceutical) composition for treating and/or preventing cancer. In the present specification, it should be noted that among the “anticancer agent”, an agent capable of killing even a CSC may be referred to as an anti-CSC agent, an anticancer agent having anti-CSC ability, or the like. It should be noted that in the present invention, “prevention (preventing)” is an action to administer an active ingredient of the present invention to a healthy human that does not develop a disease, and a purpose thereof is, for example, to prevent the onset of a disease. “Treatment (treating)” is an action to administer an active ingredient of the present invention to a person (patient) diagnosed as developing a disease by a medical doctor, and a purpose thereof is, for example, to alleviate the disease and symptoms, to inhibit the growth of carcinoma, or to return it to a state prior to the onset of the disease. In addition, even when the purpose of administration is to prevent a disease or symptoms from deteriorating or carcinoma from growing, if it is administered to a patient, it is an action for therapy.

When the present compound is administered, the amount of the compound used varies depending on symptoms, age, administration method, and the like. For example, in the case of oral administration, it is desirable to administer to an adult 0.01 mg as the lower limit (preferably 1 mg) and 5000 mg as the upper limit (preferably 500 mg) once or in several batches daily depending on the symptoms. In the case of intravenous injection, an effect is expected by administering to an adult 0.01 mg as the lower limit (preferably 0.1 mg) and 1000 mg as the upper limit (preferably 30 mg) once or in several batches daily depending on the symptoms. Examples of its administration schedule include single-dose administration, once a day administration for three days in a row, and the like. Further, each administration described above can be repeated at intervals of about 7 days to about 60 days.

The present compound can be administered orally or parenterally (e.g., intravenous, subdermal, or intramuscular injection, ocular administration, transrectally, percutaneously, transnasally, and the like). For oral administration, for example, tablets, capsules, pills, granules, powders, solutions, suspensions, and the like can be used. In addition, for parenteral administration, injections, eye drops, suppositories, patches, poultices, lotions, creams, and the like can be used. These preparations comprise the present compound and pharmaceutically acceptable additives, and are produced using conventional known techniques.

More specifically, depending on parenteral or oral administration, the present compound can be formed into a preparation using a suitable dosage form, and administrated. Examples of the dosage form include, but are not limited to, tablets, capsules, powders, granules, solutions, suspensions, injections, patches, poultices, and the like. The preparation is produced according to a known method using a pharmaceutically acceptable additive.

As additives, excipients, disintegrants, binders, fluidizers, lubricants, coating agents, solvents, solubilizing agents, thickeners, dispersants, stabilizers, sweeteners, flavors, and the like can be used in accordance with a purpose. Specific examples thereof include lactose, mannitol, crystalline cellulose, low-substituted hydroxypropylcellulose, corn starch, partly pregelatinized starch, carmellose calcium, croscarmellose sodium, hydroxypropyl cellulose, hydroxypropyl methylcellulose, polyvinyl alcohol, magnesium stearate, sodium stearyl fumarate, polyethylene glycol, propylene glycol, titanium oxide, talc, and the like.

Cancer can more effectively be prevented and/or treated by combining one to three types selected from the group consisting of: (1) administering an effective amount of the present compound; and (2)(i) administering an effective amount of another anticancer agent, (ii) administering an effective amount of a hormonal therapeutic agent; and (iii) non-pharmacological therapy. Examples of non-pharmacological therapy include surgery, radiotherapy, gene therapy, thermotherapy, cryotherapy, laser cauterization therapy, and the like. Two or more types of these also can be combined.

The present compound, even when used as a single agent, exhibits excellent anticancer effect, and further the combination use with one or several of the above-described combination drugs (polypharmacy) can further enhance its effect or improve the QOL of a patient. Hereinafter, a drug that can be used in combination with the present compound is abbreviated as a “combination drug”.

Examples of the combination drug include hormonal therapeutic agents, chemotherapeutic agents, immunotherapeutic agents, pharmaceutical agents to inhibit the activity of a cell growth factor and a receptor thereof, and the like.

Examples of “hormonal therapeutic agents” include Fosfestrol, Diethylstilbestrol, Chlorotrianisene, Medroxyprogesterone acetate, Megestrol acetate, Chlormadinone acetate, Cyproterone acetate, Danazol, Dienogest, Asoprisnil, Allylestrenol, Gestrinone, Nomegestrol, Tadenan, Mepartricin, Raloxifene, Ormeloxifene, Levormeloxifene, antiestrogen (e.g., Tamoxifen citrate, Toremifene citrate, and the like), pill preparation, Mepitiostane, Testololactone, aminoglutethimide, LH-RH derivatives (LH-RH agonist (e.g., Goserelin acetate, Buserelin, Leuprorelin, and the like), LH-RH antagonist), Droloxifene, Epitiostanol, ethynyl estradiol sulfonate, aromatase inhibitor (e.g., Fadrozole hydrochloride, Anastrozole, Letrozole, exemestane, vorozole, formestane, and the like), anti-androgen (e.g., Flutamide, Bicalutamide, Nilutamide, and the like), adrenocortical hormone type pharmaceutical agent (e.g., Dexamethasone, Prednisolone, Betamethasone, Triamcinolone, and the like), androgen synthesis inhibitor (e.g., Abiraterone and the like), retinoid and a pharmaceutical agent to delay the metabolism of retinoid (e.g., Liarozole and the like), and the like.

As “chemotherapeutic agents”, for example, alkylating agents, antimetabolites, anticancer antibiotics, plant-derived anticancer agents, other chemotherapeutic agents, and the like are used. Representative examples are described below.

Examples of “alkylating agents” include Nitrogen mustard, Nitrogen mustard N-oxide hydrochloride, Chlorambucil, Cyclophosphamide, Ifosfamide, Thiotepa, Carboquone, Improsulfan tosylate, Busulfan, Nimustine hydrochloride, Mitobronitol, Melphalan, Dacarbazine, Ranimustine, Estramustine phosphate sodium, Triethylenemelamine, Carmustine, Lomustine, Streptozocin, Pipobroman, Etoglucide, Carboplatin, Cisplatin, miriplatin, Nedaplatin, Oxaliplatin, Altretamine, Ambamustine, Dibrospidium chloride, Fotemustine, Prednimustine, Pumitepa, Ribomustin, Temozolomide, Treosulfan, Trofosfamide, Zinostatin stimalamer, Adozelesin, Cystemustine, Bizelesin, and DDS preparations thereof, and the like.

Examples of “antimetabolites” include Mercaptopurine, 6-Mercaptopurine riboside, Thioinosine, Methotrexate, Pemetrexed, Enocitabine, Cytarabine, Cytarabin ocfosfate, Ancitabine hydrochloride, 5-FU type pharmaceutical agent (e.g., Fluorouracil, Tegafur, UFT, Doxifluridine, Carmofur, Galocitabine, Emitefur, Capecitabine, and the like), Aminopterin, Nelarabine, Leucovorin calcium, tabloid, Butocin, calcium folinate, calcium levofolinate, Cladribine, Emitefur, Fludarabine, Gemcitabine, hydroxycarbamide, Pentostatin, Piritrexim, Idoxuridine, Mitoguazone, Tiazofurin, Ambamustine, Bendamustine, and DDS preparations thereof, and the like.

Examples of “anticancer antibiotics” include Actinomycin D, Actinomycin C, Mitomycin C, Chromomycin A3, Bleomycin hydrochloride, Bleomycin sulfate, Peplomycin sulfate, Daunorubicin hydrochloride, Doxorubicin hydrochloride, Aclarubicin hydrochloride, Pirarubicin hydrochloride, Epirubicin hydrochloride, Neocarzinostatin, Mithramycin, Sarkomycin, Carzinophilin, Mitotane, Zorubicin hydrochloride, Mitoxantrone hydrochloride, Idarubicin hydrochloride, and DDS preparations thereof, and the like.

Examples of “plant-derived anticancer agents” include Etoposide, Etoposide phosphate, Vinblastine sulfate, Vincristine sulfate, Vindesine sulfate, Teniposide, Paclitaxel, Docetaxel, DJ-927, Vinorelbine, Irinotecan, Topotecan, and DDS preparations thereof, and the like.

Examples of “other chemotherapeutic agents” include Sobuzoxane and the like.

Examples of “immunotherapeutic agents (BRM)” include Picibanil, Krestin, Sizofiran, Lentinan, Ubenimex, interferon, interleukin, macrophage colony stimulating factor, granulocyte-colony stimulating factor, Erythropoietin, Lymphotoxin, BCG vaccine, Corynebacterium parvum, Levamisole, polysaccharide K, Procodazole, anti-CTLA4 antibody, PD-1 antibody, Toll-like Receptors agonist (e.g., TLR7 agonist, TLR8 agonist, TLR9 agonist, and the like).

A cell growth factor in a pharmaceutical agent to inhibit the activity of the cell growth factor and a receptor thereof may be any substance as long as it promotes cell growth. Generally, it includes a factor that is a peptide having a molecular weight of 20,000 or less and exhibits an effect at a low concentration by binding with a receptor. Specifically, it includes EGF (epidermal growth factor) or substances having substantially the same activity thereas (e.g., TGFalpha and the like), insulin or substances having substantially the same activity thereas (e.g., insulin, IGF (insulin-like growth factor)-1, IGF-2, and the like), FGF (fibroblast growth factor) or substances having substantially the same activity thereas (e.g., acidic FGF, basic FGF, KGK (keratinocyte growth factor), FGF-10, and the like), and other cell growth factors (e.g., CSF (colony stimulating factor), EPO (erythropoietin), IL-2 (interleukin-2), NGF (nerve growth factor), PDGF (platelet-derived growth factor), TGF-beta (transforming growth factor beta), HGF (hepatocyte growth factor), VEGF (vascular endothelial growth factor), heregulin, angiopoietin, and the like).

The period of administration of the present compound and a combination pharmaceutical agent is not limited, and these may be administered concurrently or at intervals to a subject to be administered. In addition, a mixture of the present compound and a combination pharmaceutical agent may be made. The dosage of a combination pharmaceutical agent can be appropriately selected using a clinically used dose as criteria. In addition, the mixing ratio of the present compound and a combination pharmaceutical agent can be appropriately selected depending on a subject to be administered, an administration route, target disease, symptoms, combinations, and the like. For example, when a subject to be administered is a human, 0.01 to 100 parts by weight of a combination pharmaceutical agent may be used relative to one part by weight of the present compound. In addition, for purpose of inhibiting its side effect, they can be used in combination with a pharmaceutical agent (a combination pharmaceutical agent) such as an antiemetic agent, a sleep-inducing agent, an anticonvulsant, and the like.

### EXAMPLES

Hereinafter, the present invention is more specifically described with reference examples, Examples, and test examples. However, the technical scope of the present invention is not limited to these examples or the like. Compounds are identified by proton nuclear magnetic resonance spectrum (¹H-NMR), high performance liquid chromatography mass spectrometry (LC-MS), and the like.

Proton nuclear magnetic resonance spectra were measured using a FT-NMR measurement device (400 MHz) made by JEOL Ltd. In proton nuclear magnetic resonance spectra, tetramethylsilane was used as a standard material to describe a chemical shift value in δ value (ppm). With regard to a high performance liquid chromatography mass spectrometry, a LC-MS measurement device made by Waters Corporation was used to carry out measurement. Mass analysis was performed in an electrospray ionization method (ESI).

A LC/MS analysis condition for identification of a compound is as follows.

### LC/MS Measurement Method:

Detection apparatus: ACQUITY (registered trademark) SQ deteceter (Waters Corporation)
HPLC: ACQUITY UPLC (registered trademark) system
Column: Waters ACQUITY UPLC (registered trademark) BEH C18 (1.7 μm, 2.1 mm×30 mm)
Solvent: Solution A: 0.06% formic acid/H₂O, Solution B: 0.06% formic acid/MeCN
Gradient condition: 0.0-1.3 min Linear gradient from B 2% to 96%
Flow rate: 0.8 mL/min

### UV: 220 nm and 254 nm.

With regard to raw material compounds, reaction reagents, and solvents, unless specifically stated otherwise, those commercially available were used or those produced in accordance with a known method were used.

The present compound represented by formula (I) can be produced from a known compound by appropriately combining a method shown in the above-described methods 1 to 3, a similar method as the above-described production methods, or a well-known synthesis method to those skilled in the art. It should be noted that compound names shown in the following reference examples and examples do not always follow the IUPAC nomenclature.

In addition, the following abbreviations are sometimes used to simplify a description in the present specification.

Me: a methyl group,
Ph: a phenyl group,
THF: tetrahydrofuran,
DBU: 1,8-diazabicyclo[5.4.0]-7-undecene,
HPLC: high performance liquid chromatography,
J: coupling constant,
s: singlet,
d: doublet,
t: triplet.

### Reference Example 1

### 5-Chloro-8-hydroxynaphthalene-1,4-dione

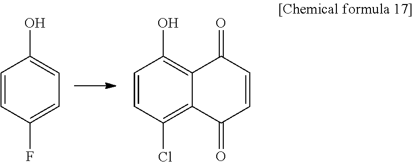 [SMILES: Oc1ccc(F)cc1 ; O=C1C=CC(=O)c2c(Cl)ccc(O)c21]

Aluminum chloride (24.4 g) and sodium chloride (4.88 g) were placed in a reaction vessel, and then the reaction mixture was heated at 180° C. To a dissolved reaction solution, 4-fluorophenol (732 mg) and maleic anhydride (2.43 g) were added, and then the reaction solution was heated and stirred at 180° C. for 10 minutes, and was poured into ice/concentrated hydrochloric acid. After collection by filtration of the reaction solution, water was added to the obtained solid, followed by extraction with chloroform. The organic layer was washed with saturated saline, dried over magnesium sulfate, filtered, and then concentrated. The resulting residue was purified by silica gel chromatography (ethyl acetate/hexane=1/9 to 1/1) to yield a solid (0.45 g).

¹H-NMR (CDCl₃) δ: 12.61 (1H, s), 7.62 (1H, d, J=12.4 Hz), 7.22 (1H, d, J=12.4 Hz), 6.93 (2H, s).

### Reference Example 2

### 8-Chloro-2-(dimethylamino)-5-hydroxynaphthalene-1,4-dione

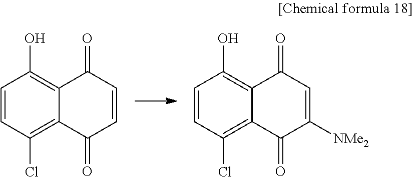 [SMILES: O=C1C=CC(=O)c2c(Cl)ccc(O)c21 ; CN(C)C1=CC(=O)c2c(O)ccc(Cl)c2C1=O]

To a toluene solution of the compound (0.42 g) obtained in Reference example 1, a solution of dimethylamine in THF (2 mol/L, 1.64 mL) was added. The reaction mixture was then stirred at −78° C. for an hour and a half. The reaction solution was filtered through Celite, and then the resulting residue was purified by silica gel chromatography (ethyl acetate/hexane=1/9 to 1/1) to yield a solid (0.23 g).

¹H-NMR (CDCl₃) δ: 13.49 (1H, s), 7.41 (1H, d, J=12.0 Hz), 7.09 (1H, d, J=12.0 Hz), 5.64 (1H, s), 3.20 (6H, s).

### Reference Example 3

### 8-Chloro-2,5-dihydroxynaphthalene-1,4-dione

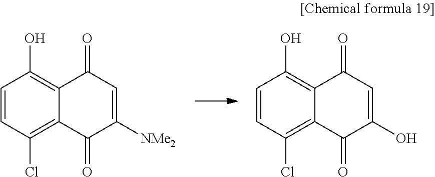 [SMILES: CN(C)C1=CC(=O)c2c(O)ccc(Cl)c2C1=O ; O=C1C=C(O)C(=O)c2c(Cl)ccc(O)c21]

To the compound (0.22 g) obtained in Reference example 2, water (5 mL) and hydrochloric acid (5 mol/L, 5 mL) were added. The reaction mixture was stirred at 90° C. for two and a half hours. The reaction solution was extracted with chloroform, and then the organic layer was washed with saturated saline, dried over magnesium sulfate, filtered, and concentrated to yield a solid (0.24 g).

¹H-NMR (CDCl₃) δ: 13.00 (1H, s), 7.68 (1H, s), 7.51 (1H, d, J=12.4 Hz), 7.22 (1H, d, J=12.4 Hz), 6.27 (1H, s).

### Reference Example 4

### 2-Acetyl-8-chloro-5-hydroxy-2,3-dihydronaphtho[2,3-b]furan-4,9-dione

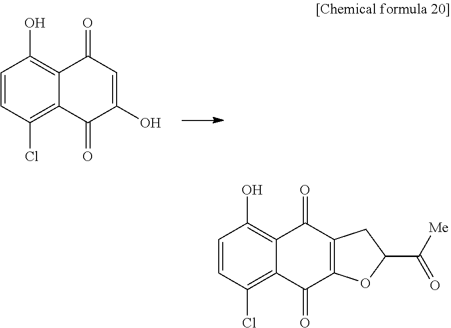 [SMILES: O=C1C=C(O)C(=O)c2c(Cl)ccc(O)c21 ; CC(=O)C1CC2=C(O1)C(=O)c1c(Cl)ccc(O)c1C2=O]

To a solution of methyl vinyl ketone (403 mg) in pentane (3 mL) at −15° C. was added a solution of bromine (930 mg) in pentane (3 mL). The reaction solution was stirred for 30 minutes, and then concentrated. A THF solution (5 mL) of the compound (215 mg) obtained in Reference example 3 was cooled to 0° C. To this solution, the reaction solution concentrate prepared by the above method, and DBU (1.03 ml) were added. It was then stirred at room temperature overnight. After collection by filtration, water was added to the obtained solid, followed by extraction with chloroform. The organic layer was washed with saturated saline, dried over magnesium sulfate, filtered, and concentrated. The resulting residue was purified by silica gel chromatography (ethyl acetate/hexane=1/9 to 2/1) to yield an orange solid (132 mg).

¹H-NMR (CDCl₃) δ: 12.82 (1H, s), 7.52 (1H, d, J=12.4 Hz), 7.19 (1H, d, J=12.4 Hz), 5.28 (1H, t, J=12.8 Hz), 3.38 (2H, d, J=12.8 Hz), 2.37 (3H, s).

### Reference Example 5

### 6-Bromo-5-hydroxynaphthalene-1,4-dione

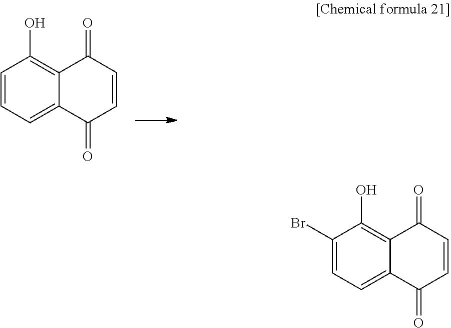 [SMILES: O=C1C=CC(=O)c2c(O)cccc21 ; O=C1C=CC(=O)c2c1ccc(Br)c2O]

To a solution of 5-hydroxynaphthalene-1,4-dione (1.01 g) in acetic acid (25 mL) was added bromine (0.36 g) and sodium acetate (0.95 g). The reaction mixture was stirred at room temperature overnight: An aqueous saturated sodium hydrogen carbonate solution was added to the reaction solution, and then it was extracted with ethyl acetate. The organic layer was washed with saturated saline, dried over magnesium sulfate, filtered, and then concentrated. The resulting residue was purified by silica gel chromatography (ethyl acetate/hexane=1/9 to 1/1) to yield an orange solid (0.39 g).

¹H-NMR (CDCl₃) δ: 12.58 (1H, s), 7.94 (1H, d, J=8.0 Hz), 7.52 (1H, d, J=8.0 Hz), 6.99 (2H, s).

### Reference Examples 6 and 7

### 6-Bromo-2-(dimethylamino)-5-hydroxynaphthalene-1,4-dione (Reference Example 6)

### 7-Bromo-2-(dimethylamino)-8-hydroxynaphthalene-1,4-dione (Reference Example 7)

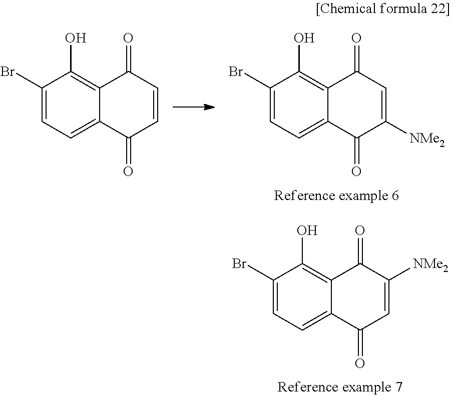 [SMILES: O=C1C=CC(=O)c2c1ccc(Br)c2O ; CN(C)C1=CC(=O)c2c(ccc(Br)c2O)C1=O ; CN(C)C1=CC(=O)c2ccc(Br)c(O)c2C1=O]

Reference example 6 (4.58 g) and Reference example 7 (1.46 g) were obtained by reacting the compound (15.4 g) obtained in Reference example 5 similarly as Reference example 2.

Reference example 6: ¹H-NMR (CDCl₃) δ: 13.87 (1H, s), 7.74 (1H, d, J=8.0 Hz), 7.38 (1H, d, J=8.0 Hz), 5.73 (1H, s), 3.27 (6H, s), (LC-MS: [M+H]⁺/Rt (min))=296/0.99
Reference example 7: (LC-MS: [M+H]⁺/Rt (min))=296/0.91

### Reference Example 8

### 6-Bromo-2,5-dihydroxynaphthalene-1,4-dione

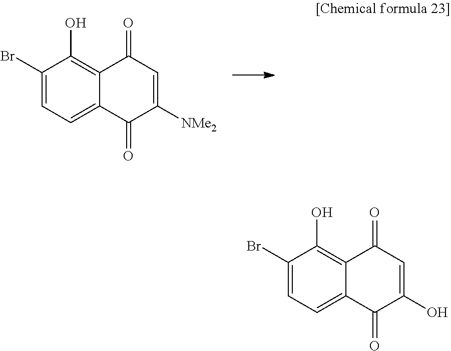 [SMILES: CN(C)C1=CC(=O)c2c(ccc(Br)c2O)C1=O ; O=C1C(O)=CC(=O)c2c1ccc(Br)c2O]

A yellow solid (4.1 g) was obtained by reacting the compound (4.58 g) obtained in Reference example 6 similarly as Reference example 3.

(LC-MS: [M+H]⁺/Rt (min))=269/0.83

### Reference Example 9

### 7-Bromo-2,8-dihydroxynaphthalene-1,4-dione

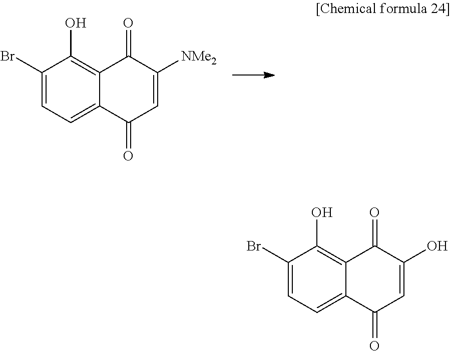 [SMILES: CN(C)C1=CC(=O)c2ccc(Br)c(O)c2C1=O ; O=C1C=C(O)C(=O)c2c1ccc(Br)c2O]

A yellow solid (1.11 g) was obtained by reacting the compound (1.46 g) obtained in Reference example 7 similarly as Reference example 3.

(LC-MS: [M+H]⁺/Rt (min))=269/0.77

### Reference Example 10

### 2-Acetyl-6-bromo-5-hydroxy-2,3-dihydronaphtho[2,3-b]furan-4,9-dione

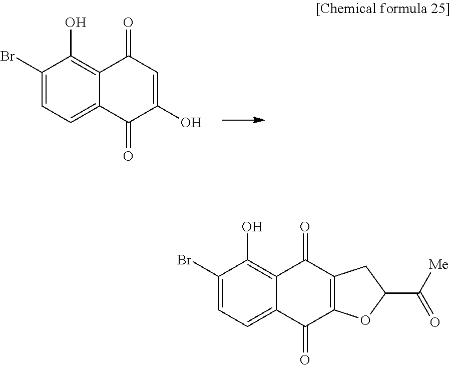 [SMILES: O=C1C(O)=CC(=O)c2c1ccc(Br)c2O ; CC(=O)C1CC2=C(O1)C(=O)c1ccc(Br)c(O)c1C2=O]

A yellow solid (0.3 g) was obtained by reacting the compound (1.0 g) obtained in Reference example 8 similarly as Reference example 4.

(LC-MS: [M+H]⁺/Rt (min))=337/0.83

### Example 1

### 2-Acetyl-8-chloro-5-hydroxynaphtho[2,3-b]furan-4,9-dione

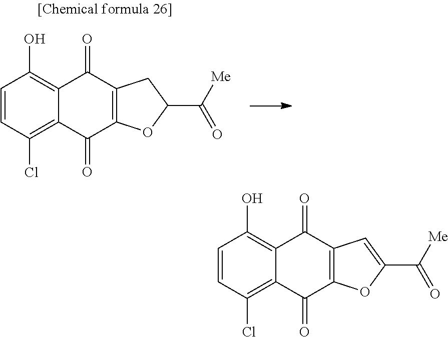 [SMILES: CC(=O)C1CC2=C(O1)C(=O)c1c(Cl)ccc(O)c1C2=O ; CC(=O)c1cc2c(o1)C(=O)c1c(Cl)ccc(O)c1C2=O]

To a toluene (10 mL) solution of the compound (130 mg) obtained in Reference example 4, manganese dioxide (386 mg) was added. The reaction mixture was stirred at 110° C. for twelve and a half hours. After completion of heating, the reaction solution was filtered through Celite with ethyl acetate. The filtrate was concentrated under reduced pressure, and then the resulting residue was purified by silica gel chromatography (ethyl acetate/hexane=1/9 to 2/1) to yield a yellow solid (18 mg).

¹H-NMR (CDCl₃) δ: 12.85 (1H, s), 7.63 (1H, d, J=12.0 Hz), 7.56 (1H, s), 7.26 (1H, d, J=12.0 Hz), 2.65 (3H, s).

### Example 2a

### 2-Acetyl-6-bromo-5-hydroxynaphtho[2,3-b]furan-4,9-dione

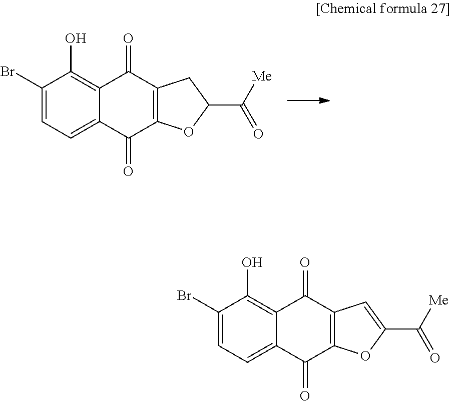 [SMILES: CC(=O)C1CC2=C(O1)C(=O)c1ccc(Br)c(O)c1C2=O ; CC(=O)c1cc2c(o1)C(=O)c1ccc(Br)c(O)c1C2=O]

To a solution of Reference example 10 (1.5 g) in acetic acid (30 mL) was added pyridinium bromide perbromide (1.28 g). The reaction mixture was stirred at 70° C. for 2 hours. Water was added to the reaction solution, and then it was extracted with ethyl acetate. The organic layer was washed with saturated saline, dried over magnesium sulfate, filtered, and concentrated. Ethyl acetate (50 mL) was added to the resulting solid, and then stirred and heated at 80° C. The solid was collected by filtration, and then washed with ethyl acetate. The same operation was repeated three times, and then the solid was collected and dried to yield an orange solid (865 mg).

¹H-NMR (CDCl₃) δ: 12.79 (1H, s), 7.94 (1H, d, J=8.0 Hz), 7.67 (1H, d, J=8.0 Hz), 7.58 (1H, s), 2.64 (3H, s).

### Example 2b

### 2-Acetyl-6-bromo-5-hydroxynaphtho[2,3-b]furan-4,9-dione

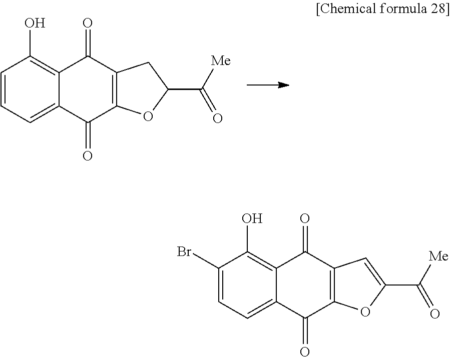 [SMILES: CC(=O)C1CC2=C(O1)C(=O)c1cccc(O)c1C2=O ; CC(=O)c1cc2c(o1)C(=O)c1ccc(Br)c(O)c1C2=O]

To a solution of a 2-acetyl-5-hydroxy-2,3-dihydronaphtho[2,3-b]furan-4,9-dione compound (23 mg) in acetic acid (1 mL) was added bromine (22 mg) and sodium acetate (22 mg). The reaction mixture was stirred at 70° C. for 5 hours. An aqueous saturated sodium hydrogen carbonate solution was added to the reaction solution, and then it was extracted with ethyl acetate. The organic layer was washed with saturated saline, dried over magnesium sulfate, filtered, and then concentrated. The resulting residue was purified by silica gel chromatography (ethyl acetate/hexane=1/9 to 2/1) to yield a solid (9.9 mg) as an orange solid.

¹H-NMR (CDCl₃) δ: 12.79 (1H, s), 7.94 (1H, d, J=8.0 Hz), 7.67 (1H, d, J=8.0 Hz), 7.58 (1H, s), 2.64 (3H, s).

### Example 3

### 2-Acetyl-7-bromo-8-hydroxynaphtho[2,3-b]furan-4,9-dione

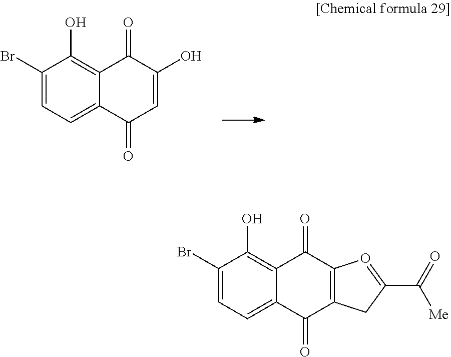

A yellow solid (0.12 g) was obtained by reacting the compound (1.1 g) obtained in Reference example 9 similarly as Reference example 10 and Example 2a.

(LC-MS: [M+H]⁺/Rt (min))=337/0.91

### Example 4

### 2-Acetyl-6-butyl-5-hydroxynaphtho[2,3-b]furan-4,9-dione

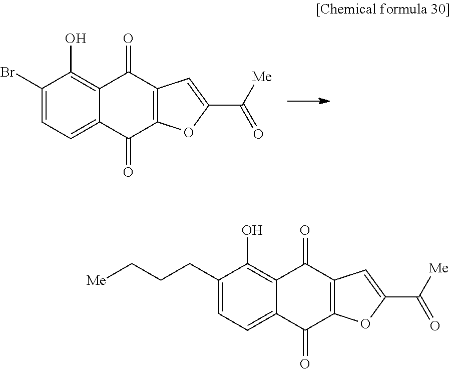 [SMILES: CC(=O)c1cc2c(o1)C(=O)c1ccc(Br)c(O)c1C2=O ; CCCCc1ccc2c(c1O)C(=O)c1cc(C(C)=O)oc1C2=O]

To a toluene (0.5 mL) solution of the compound (20 mg) obtained in Example 2b was added 2-(tri-n-butylstannyl)oxazole (0.043 mL), tetrakis(triphenylphosphine)palladium (0) (13.8 mg). The reaction mixture was stirred at 80° C. for 2 hours. An aqueous saturated sodium hydrogen carbonate solution was added to the reaction solution, and then it was extracted with ethyl acetate. The organic layer was washed with saturated saline, dried over magnesium sulfate, filtered, and then concentrated. The resulting residue was purified by silica gel chromatography (ethyl acetate/hexane=1/9 to 2/1) to yield a solid (2.0 mg) as an orange solid.

(LC-MS: [M+H]⁺/Rt (min))=313/1.21

### Example 5

### 2-Acetyl-5-hydroxy-6-(4-methoxyphenyl)naphtho[2,3-b]furan-4,9-dione

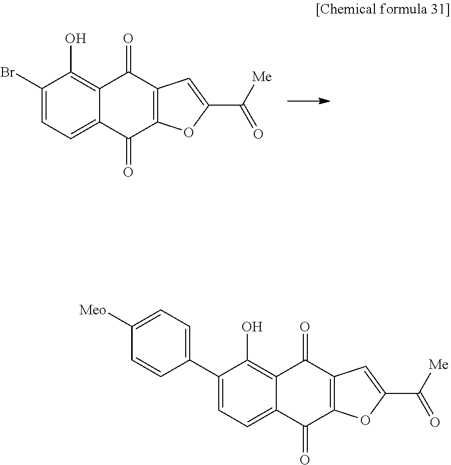

To a 1,4-dioxane (1.5 mL) solution of the compound (30 mg) obtained in Example 2a was added 4-methoxyphenylboronic acid (27.2 mg), tetrakis(triphenylphosphine)palladium (0) (20.7 mg), and 2 mol/L aqueous sodium carbonate solution (0.13 mL). The reaction mixture was stirred at 80° C. for 1.5 hours under microwave irradiation. Water was added to the reaction solution, and then it was extracted with ethyl acetate. The organic layer was washed with saturated saline, dried over magnesium sulfate, filtered, and then concentrated. The resulting residue was purified by reverse-phase preparative HPLC to yield a solid (10.5 mg).

(LC-MS: [M+H]⁺/Rt (min))=363/1.13

### Example 6

### 2-Acetyl-5-hydroxy-6-(2-methoxypyrimidin-5-yl)naphtho[2,3-b]furan-4,9-dione

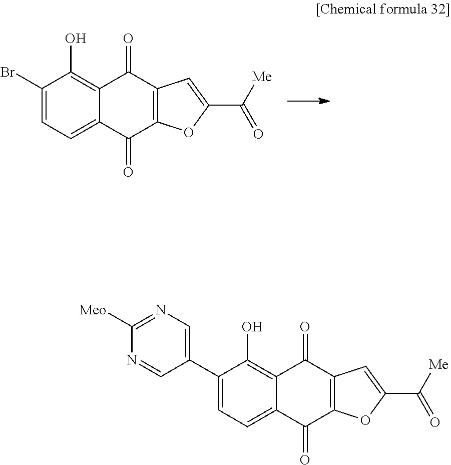

A yellow solid (1.2 mg) was obtained by reacting the compound (50 mg) obtained in Example 2a in accordance with the method described in Example 5.

(LC-MS: [M+H]⁺/Rt (min))=365/0.92

### Example 7

### 2-Acetyl-6-bromo-5-(methoxymethoxy)naphtho[2,3-b]furan-4,9-dione

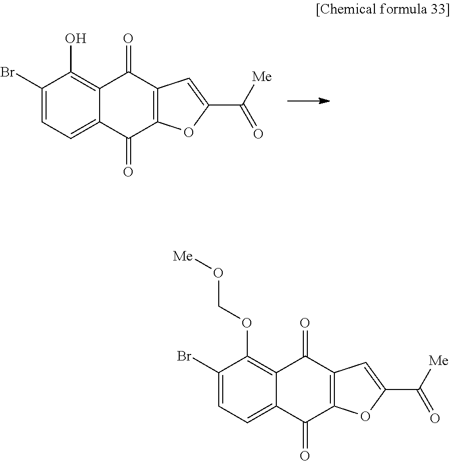 [SMILES: CC(=O)c1cc2c(o1)C(=O)c1ccc(Br)c(O)c1C2=O ; COCOc1c(Br)ccc2c1C(=O)c1cc(C(C)=O)oc1C2=O]

To a methylene chloride (10 mL) solution of the compound (35 mg) obtained in Example 2a was added chloromethyl methyl ether (13 uL) and triethylamine (0.15 uL). The reaction mixture was stirred at 0° C. for 2 hours. Water was added to the reaction solution, and then it was extracted with ethyl acetate. The organic layer was washed with saturated saline, dried over magnesium sulfate, filtered, and then concentrated. The resulting residue was purified by silica gel chromatography (ethyl acetate/chloroform=1/1) to yield a solid (10.0 mg) as an orange solid.

(LC-MS: [M+H]⁺/Rt (min))=380/0.96

### Example 8

### 2-Acetyl-6-bromo-4,9-dioxo-4,9-dihydronaphtho[2,3-b]furan-5-yl benzoate

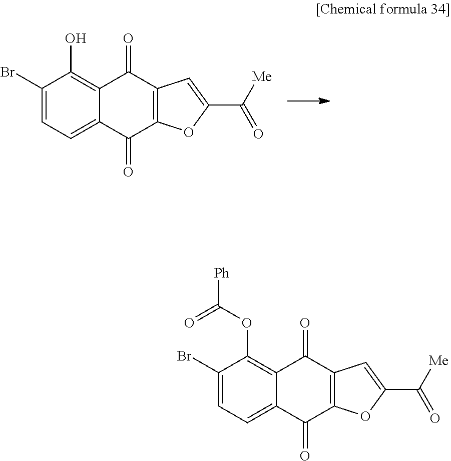 [SMILES: CC(=O)c1cc2c(o1)C(=O)c1ccc(Br)c(O)c1C2=O ; CC(=O)c1cc2c(o1)C(=O)c1ccc(Br)c(OC(=O)c3ccccc3)c1C2=O]

To a methylene chloride (10 mL) solution of the compound (35 mg) obtained in Example 2a was added benzoyl chloride (18 uL) and triethylamine (0.15 uL). The reaction mixture was stirred at 0° C. for 2 hours. Water was added to the reaction solution, and then it was extracted with ethyl acetate. The organic layer was washed with saturated saline, dried over magnesium sulfate, filtered, and then concentrated. The resulting residue was purified by silica gel chromatography (ethyl acetate/chloroform=1/1) to yield a solid (11.0 mg) as an orange solid.

(LC-MS: [M+H]⁺/Rt (min))=440/1.14

### Example 9

### 2-Acetyl-6-bromo-4,9-dioxo-4,9-dihydronaphtho[2,3-b]furan-5-yl 2-methylpropyl carbonate

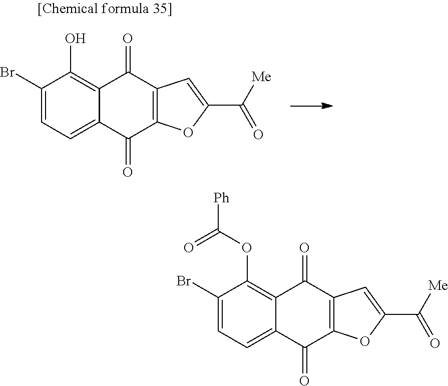 [SMILES: CC(=O)c1cc2c(o1)C(=O)c1ccc(Br)c(O)c1C2=O ; CC(=O)c1cc2c(o1)C(=O)c1ccc(Br)c(OC(=O)c3ccccc3)c1C2=O]

To a methylene chloride (10 mL) solution of the compound (35 mg) obtained in Example 2a was added isobutyl chloroformate (15 uL) and triethylamine (0.15 uL). The reaction mixture was stirred at 0° C. for 2 hours. Water was added to the reaction solution, and then it was extracted with ethyl acetate. The organic layer was washed with saturated saline, dried over magnesium sulfate, filtered, and then concentrated. The resulting residue was purified by silica gel chromatography (ethyl acetate/chloroform=1/1) to yield a solid (13.0 mg) as an orange solid.

(LC-MS: [M+H]⁺/Rt (min))=436/1.13

The following compounds can be produced according to similar methods as the above-described Reference examples 1, 2, 3, and 4, and the above-described Example 1:
2-acetyl-6-chloro-5-hydroxynaphtho[2,3-b]furan-4,9-dione;
2-acetyl-7-chloro-5-hydroxynaphtho[2,3-b]furan-4,9-dione;
2-acetyl-5-chloro-8-hydroxynaphtho[2,3-b]furan-4,9-dione;
2-acetyl-6-chloro-8-hydroxynaphtho[2,3-b]furan-4,9-dione;
2-acetyl-7-chloro-8-hydroxynaphtho[2,3-b]furan-4,9-dione;
2-acetyl-8-chloro-7-hydroxynaphtho[2,3-b]furan-4,9-dione;
2-acetyl-6-chloro-7-hydroxynaphtho[2,3-b]furan-4,9-dione;
2-acetyl-5-chloro-7-hydroxynaphtho[2,3-b]furan-4,9-dione;
2-acetyl-5-chloro-6-hydroxynaphtho[2,3-b]furan-4,9-dione;
2-acetyl-7-chloro-6-hydroxynaphtho[2,3-b]furan-4,9-dione; and
2-acetyl-8-chloro-6-hydroxynaphtho[2,3-b]furan-4,9-dione.

### Test Example 1: Proliferation Inhibition Assay of Cancer Cells

HCT-116 cells were obtained from American Type Culture Collection (ATCC). HCT-116 cells were cultured at 37° C. in the presence of 5% CO₂ using McCoy's 5a medium containing 10% fetal bovine serum (FBS) and 1% penicillin/streptomycin. After cells were seeded at 300-600 cells/well onto μClear-plate black 384 well (Greiner bio-one Cat. No. 781091), a test substance was added such that the final concentration of DMSO is 0.1%, and cultured for 4 days. The number of living cells was then counted using Cell Titer-Glo (registered trademark) Luminescent Cell Viability Assay (Promega) to calculate a concentration for 50% inhibition of cell proliferation of respective test substances (Bulk IC₅₀ value; μM).

TABLE 1

| | IC₅₀ (μM) |
|---|---|
| Example | HCT |
| 1 | 0.63 |
| 2 | 0.63 |
| 3 | 4.93 |

### Test Example 2. Inhibition Assay of Cancer Cell Sphere-Forming Ability

HCT-116 cells were obtained from American Type Culture Collection (ATCC). HCT-116 cells were cultured at 37° C. in the presence of 5% CO₂ using McCoy's 5a medium containing 10% fetal bovine serum (FBS) and 1% penicillin/streptomycin. The HCT-116 cells were seeded onto 384 Well Black Clear Bottom Ultra-Low Attachment Microplate (Corning Cat. No. 3827) using DMEM/F12 medium containing 2% B27 supplement (GIBCO), 20 ng/mL epidermal growth factor (EGF) (peprotech), 10 ng/mL basic fibroblast growth factor (bFGF) (peprotech), 5 μg/mL insulin (Sigma), and 1% penicillin/streptomycin to be 350-800 cells/well. A test substance was added so that the final concentration of DMSO is 0.1%, and then cultured for 4 days. The number of living cells was then counted using Cell Titer-Glo (registered trademark) Luminescent Cell Viability Assay (Promega) to calculate the concentration for 50% inhibition of cell proliferation of respective test substances (Sphere IC₅₀ value; μM) (this test is a method which evaluates the functions of cancer stem cells (see Non Patent Literature 4)).

TABLE 2

| | IC₅₀ (μM) |
|---|---|
| Example | HCT |
| 1 | 0.24 |
| 2 | 5.12 |
| 3 | 6.39 |

As described above, the present invention is illustrated by preferable embodiments of the present invention. However, it will be understood that the scope of the present invention should be interpreted only by the claims. It will be understood that the contents of patents, patent applications, and literatures cited in the present specification should be incorporated by reference to the present specification as if their contents per se are specifically described in the present specification.

### INDUSTRIAL APPLICABILITY

The present invention provides a compound useful as a novel antitumor agent targeting a CSC that is important in continuous proliferation of malignant tumor, metastasis and recurrence of cancer, and its resistance to an antitumor agent; a medicament comprising the compound as an active ingredient; a pharmaceutical composition; and an antitumor agent; as well as a method of treating cancer and/or a method of preventing cancer.

1. A compound represented by formula (I): 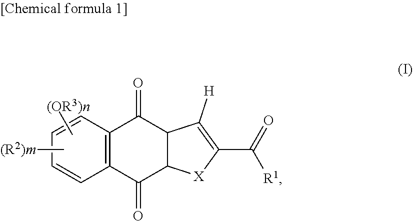
or a pharmaceutically acceptable salt thereof, wherein
X is an oxygen atom or sulfur atom;
R¹ is a hydrogen atom, a cyano group, a trifluoromethyl group, a trifluoromethoxy group, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, an optionally substituted 5 to 12-membered monocyclic or polycyclic heteroaryl group, —OR⁴, or —SR⁴;
each R² is independently a halogen atom, a cyano group, a nitro group, a trifluoromethyl group, a trifluoromethoxy group, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted C₁₋₆alkylcarbonyl group, an optionally substituted C₃₋₁₀cycloalkylcarbonyl group, an optionally substituted C₆₋₁₀arylcarbonyl group, an optionally substituted 5 to 12-membered monocyclic or polycyclic heteroarylcarbonyl group, an optionally substituted 3 to 10-membered saturated heterocyclic group, an optionally substituted C₆₋₁₀aryl group, an optionally substituted 5 to 12-membered monocyclic or polycyclic heteroaryl group, an optionally substituted C₁₋₆alkylsulfonyl group, an optionally substituted C₃₋₁₀cycloalkylsulfonyl group, an optionally substituted C₆₋₁₀arylsulfonyl group, an optionally substituted aminosulfonyl group, —N(R′)₂, or —SR⁴;
each R³ is independently a hydrogen atom, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, an optionally substituted 5 to 12-membered monocyclic or polycyclic heteroaryl group, an optionally substituted C₁₋₆alkylcarbonyl group, an optionally substituted C₃₋₁₀cycloalkylcarbonyl group, an optionally substituted C₆₋₁₀arylcarbonyl group, an optionally substituted 3 to 10-membered saturated heterocyclylcarbonyl group, an optionally substituted 5 to 12-membered monocyclic or polycyclic heteroarylcarbonyl group, an optionally substituted C₁₋₆alkyloxycarbonyl group, an optionally substituted C₃₋₁₀cycloalkyloxycarbonyl group, an optionally substituted C₆₋₁₀aryloxycarbonyl group, an optionally substituted 3 to 10-membered saturated heterocyclyloxycarbonyl group, an optionally substituted 5 to 12-membered monocyclic or polycyclic heteroaryloxycarbonyl group, an optionally substituted C₁₋₆alkylaminocarbonyl group, an optionally substituted C₃₋₁₀cycloalkylaminocarbonyl group, an optionally substituted C₆₋₁₀arylaminocarbonyl group, an optionally substituted 3 to 10-membered saturated heterocyclylaminocarbonyl group, or an optionally substituted 5 to 12-membered monocyclic or polycyclic heteroarylaminocarbonyl group;
m is O, 1, 2, 3, or 4 and n is 1, 2, 3, or 4, with the proviso that the sum of m and n is 1, 2, 3, or 4; and
each R⁴ is independently a hydrogen atom, an optionally substituted C₁₋₆alkyl group, an optionally substituted C₂₋₆alkenyl group, an optionally substituted C₂₋₆alkynyl group, an optionally substituted C₃₋₁₀cycloalkyl group, an optionally substituted C₃₋₁₀cycloalkenyl group, an optionally substituted 3 to 10-membered saturated heterocyclic group, an optionally substituted C₆₋₁₀aryl group, or an optionally substituted 5 to 12-membered monocyclic or polycyclic heteroaryl group, or when two R⁴ are attached to a nitrogen atom, the two R⁴ may be taken together with the nitrogen atom to which they are attached to form a nitrogen-containing 3 to 10-membered saturated heterocyclic group,
with the proviso that the following compounds are excluded:
5-hydroxy-4,9-dioxo-4,9-dihydronaphtho[2,3-b]furan-2-carbaldehyde;
2-acetyl-5-hydroxynaphtho[2,3-b]furan-4,9-dione;
2-acetyl-8-hydroxynaphtho[2,3-b]furan-4,9-dione;
2-acetyl-6-methoxynaphtho[2,3-b]furan-4,9-dione;
2-acetyl-7-methoxynaphtho[2,3-b]furan-4,9-dione;
2-acetyl-8-methoxynaphtho[2,3-b]furan-4,9-dione;
2-acetyl-5-methoxynaphtho[2,3-b]furan-4,9-dione;
2-acetyl-8-hydroxy-7-methoxynaphtho[2,3-b]furan-4,9-dione;
2-acetyl-6,7-di methoxynaphtho[2,3-b]furan-4,9-dione;
2-acetyl-7,8-dimethoxynaphtho[2,3-b]furan-4,9-dione;
2-acetyl-5,6-dimethoxynaphtho[2,3-b]furan-4,9-dione;
2-benzoyl-5-hydroxynaphtho[2,3-b]furan-4,9-dione;
8-hydroxy-4,9-dioxo-4,9-dihydronaphtho[2,3-b]thiophene-2-carbaldehyde;
5-hydroxy-4,9-dioxo-4,9-dihydronaphtho[2,3-b]thiophene-2-carbaldehyde;
8-methoxy-4,9-dioxo-4,9-dihydronaphtho[2,3-b]thiophene-2-carbaldehyde;
8-hydroxy-4,9-dioxo-4,9-dihydronaphtho[2,3-b]thiophene-2-carboxylic acid;
2-acetyl-8-hydroxynaphtho[2,3-b]thiophene-4,9-dione;
2-acetyl-5-hydroxynaphtho[2,3-b]thiophene-4,9-dione;
2-acetyl-5,8-dihydroxynaphtho[2,3-b]thiophene-4,9-dione;
8-hydroxy-2-propanoylnaphtho[2,3-b]thiophene-4,9-dione;
2-acetyl-8-methoxynaphtho[2,3-b]thiophene-4,9-dione;
8-methoxy-4,9-dioxo-4,9-dihydronaphtho[2,3-b]thiophene-2-carboxylic acid;
methyl 8-hydroxy-4,9-dioxo-4,9-dihydronaphtho[2,3-b]thiophene-2-carboxylate;
methyl 5-hydroxy-4,9-dioxo-4,9-dihydronaphtho[2,3-b]thiophene-2-carboxylate;
8-hydroxy-2-(2-methylpropanoyl)naphtho[2,3-b]thiophene-4,9-dione;
2-acetyl-8-hydroxy-7-(hydroxymethyl)naphtho[2,3-b]thiophene-4,9-dione;
2-acetyl-5,8-dimethoxynaphtho[2,3-b]thiophene-4,9-dione;
8-methoxy-2-propanoylnaphtho[2,3-b]thiophene-4,9-dione;
ethyl 8-hydroxy-4,9-dioxo-4,9-dihydronaphtho[2,3-b]thiophene-2-carboxylate;
8-hydroxy-2-(thiophene-2-ylcarbonyl)naphtho[2,3-b]thiophene-4,9-dione;
8-methoxy-2-(2-methylpropanoyl)naphtho[2,3-b]thiophene-4,9-dione;
2-benzoyl-5-hydroxynaphtho[2,3-b]thiophene-4,9-dione;
2-benzoyl-8-hydroxynaphtho[2,3-b]thiophene-4,9-dione;
propan-2-yl 8-hydroxy-4,9-dioxo-4,9-dihydronaphtho[2,3-b]thiophene-2-carboxylate;
ethyl 8-methoxy-4,9-dioxo-4,9-dihydronaphtho[2,3-b]thiophene-2-carboxylate;
8-methoxy-2-(thiophene-2-ylcarbonyl)naphtho[2,3-b]thiophene-4,9-dione;
propan-2-yl 8-methoxy-4,9-dioxo-4,9-dihydronaphtho[2,3-b]thiophene-2-carboxylate;
2-benzoyl-8-methoxynaphtho[2,3-b]thiophene 4,9-dione;
8-hydroxy-2-(phenylacetyl)naphtho[2,3-b]thiophene-4,9-dione;
8-hydroxy-2-(4-methoxybenzoyl)naphtho[2,3-b]thiophene-4,9-dione;
2-(4-chlorobenzoyl)-8-hydroxynaphtho[2,3-b]thiophene-4,9-dione;
8-methoxy-2-(4-methoxybenzoyl)naphtho[2,3-b]thiophene-4,9-dione;
8-hydroxy-2-(3-phenylpropanoyl)naphtho[2,3-b]thiophene-4,9-dione;
8-methoxy-2-(phenylacetyl)naphtho[2,3-b]thiophene-4,9-dione;
8-methoxy-2-(3-phenylpropanoyl)naphtho[2,3-b]thiophene-4,9-dione;
8-hydroxy-2-(phenoxyacetyl)naphtho[2,3-b]thiophene-4,9-dione;
8-hydroxy-2-(pyridin-3-ylcarbonyl)naphtho[2,3-b]thiophene-4,9-dione;
2-(4-chlorobenzoyl)-8-methoxynaphtho[2,3-b]thiophene-4,9-dione;
4-methoxybenzyl 8-hydroxy-4,9-dioxo-4,9-dihydronaphtho[2,3-b]thiophene-2-carboxylate;
2-phenylethyl 8-hydroxy-4,9-dioxo-4,9-dihydronaphtho[2,3-b]thiophene-2-carboxylate;
8-methoxy-2-(phenoxyacetyl)naphtho[2,3-b]thiophene-4,9-dione;
8-methoxy-2-(pyridin-3-ylcarbonyl)naphtho[2,3-b]thiophene-4,9-dione;
4-methoxybenzyl 8-methoxy-4,9-dioxo-4,9-dihydronaphtho[2,3-b]thiophene-2-carboxylate;
8-hydroxy-2-(2-hydroxy-4-methoxybenzoyl)naphtho[2,3-b]thiophene-4,9-dione;
2-phenylethyl 8-methoxy-4,9-dioxo-4,9-dihydronaphtho[2,3-b]thiophene-2-carboxylate; and
2-(2-hydroxy-4-methoxybenzoyl)-8-methoxynaphtho[2,3-b]thiophene-4,9-dione.

2. The compound according to claim 1, or a pharmaceutically acceptable salt thereof, wherein X is an oxygen atom.

3. The compound according to claim 1, or a pharmaceutically acceptable salt thereof, wherein R¹ is a C₁₋₆alkyl group optionally substituted with one to three halogen atoms, hydroxyl groups, one or two C₁₋₆alkyloxy groups, or a 3 to 10-membered saturated heterocyclic group.

4. The compound according to claim 3, or a pharmaceutically acceptable salt thereof, wherein R¹ is a methyl group.

5. The compound according to claim 1, or a pharmaceutically acceptable salt thereof, wherein each R² is independently a halogen atom, a cyano group, an optionally substituted C₁₋₆alkyl group, an optionally substituted C₂₋₆alkenyl group, an optionally substituted C₂₋₆alkynyl group, an optionally substituted C₃₋₁₀cycloalkenyl group, an optionally substituted C₁₋₆alkylcarbonyl group, an optionally substituted C₆₋₁₀aryl group, or an optionally substituted 5 to 12-membered monocyclic or polycyclic heteroaryl group.

6. The compound according to claim 5, or a pharmaceutically acceptable salt thereof, wherein each R² is independently a halogen atom, an optionally substituted C₁₋₆alkyl group, an optionally substituted C₆₋₁₀aryl group, or an optionally substituted 5 to 12-membered monocyclic or polycyclic heteroaryl group.

7. The compound according to claim 5, or a pharmaceutically acceptable salt thereof, wherein each R² is independently a halogen atom or an optionally substituted C₁₋₆alkyl group.

8. The compound according to claim 5, or a pharmaceutically acceptable salt thereof, wherein each R² is independently a halogen atom.

9. The compound according to claim 1, or a pharmaceutically acceptable salt thereof, wherein each R³ is independently a hydrogen atom, an optionally substituted C₁₋₆alkyl group, an optionally substituted C₆₋₁₀aryl group, an optionally substituted 5 to 12-membered monocyclic or polycyclic heteroaryl group, an optionally substituted C₁₋₆alkylcarbonyl group, an optionally substituted C₆₋₁₀arylcarbonyl group, an optionally substituted C₁₋₆alkyloxycarbonyl group, an optionally substituted C₁₋₆aryloxycarbonyl group, an optionally substituted C₁₋₆alkylaminocarbonyl group, or an optionally substituted C₆₋₁₀arylaminocarbonyl group.

10. The compound according to claim 9, or a pharmaceutically acceptable salt thereof, wherein each R³ is independently a hydrogen atom, an optionally substituted C₁₋₆alkyl group, an optionally substituted C₆₋₁₀arylcarbonyl group, or an optionally substituted C₁₋₆alkyloxycarbonyl group.

11. The compound according to claim 9, or a pharmaceutically acceptable salt thereof, wherein each R³ is independently a hydrogen atom.

12. The compound according to claim 1, or a pharmaceutically acceptable salt thereof, wherein each R⁴ is independently a hydrogen atom, an optionally substituted C₁₋₆alkyl group, an optionally substituted C₃₋₁₀cycloalkyl group, an optionally substituted C₆₋₁₀aryl group, or an optionally substituted 5 to 12-membered monocyclic or polycyclic heteroaryl group, or when two R⁴ are attached to a nitrogen atom, the two R⁴ may be taken together with the nitrogen atom to which they are attached to form a nitrogen-containing 3 to 10-membered saturated heterocyclic group.

13. The compound according to claim 12, or a pharmaceutically acceptable salt thereof, wherein each R⁴ is independently a hydrogen atom or an optionally substituted C₁₋₆alkyl group, or when two R⁴ are attached to a nitrogen atom, the two R⁴ may be taken together with the nitrogen atom to which they are attached to form a nitrogen-containing 3 to 10-membered saturated heterocyclic group.

14. The compound according to claim 1, or a pharmaceutically acceptable salt thereof, wherein m and n are both 1.

15. The compound according to claim 1, or a pharmaceutically acceptable salt thereof, wherein R¹ is a methyl group; each R² is independently a halogen atom or an optionally substituted C₁₋₆alkyl group; and each R³ is independently a hydrogen atom.

16. The compound according to claim 15, or a pharmaceutically acceptable salt thereof, wherein m and n are both 1.

17. The compound according to claim 1, or a pharmaceutically acceptable salt thereof, wherein R¹ is a methyl group; each R² is independently a halogen atom; and each R³ is independently a hydrogen atom.

18. The compound according to claim 17, or a pharmaceutically acceptable salt thereof, wherein m and n are both 1.

19. The compound according to claim 1, or a pharmaceutically acceptable salt thereof, wherein the compound is selected from the group consisting of the following compounds:
2-acetoxy-8-chloro-5-hydroxynaphtho[2,3-b]furan-4,9-dione;
2-acetyl-6-bromo-5-hydroxynaphtho[2,3-b]furan-4,9-dione;
2-acetyl-7-bromo-8-hydroxynaphtho[2,3-b]furan-4,9-dione;
2-acetyl-6-butyl-5-hydroxynaphtho[2,3-b]furan-4,9-dione;
2-acetyl-5-hydroxy-6-(4-methoxyphenyl)naphtho[2,3-b]furan-4,9-dione;
2-acetyl-5-hydroxy-6-(2-methoxypyrimidin-5-yl)naphtho[2,3-b]furan-4,9-dione;
2-acetyl-6-bromo-5-(methoxymethoxy)naphtho[2,3-b]furan-4,9-dione;
2-acetyl-6-bromo-4,9-dioxo-4,9-dihydronaphtho[2,3-b]furan-5-yl benzoate;
2-acetyl-6-bromo-4,9-dioxo-4,9-dihydronaphtho[2,3-b]furan-5-yl 2-methyl propyl carbonate;
2-acetyl-6-chloro-5-hydroxynaphtho[2,3-b]furan-4,9-dione;
2-acetyl-7-chloro-5-hydroxynaphtho[2,3-b]furan-4,9-dione;
2-acetyl-5-chloro-8-hydroxynaphtho[2,3-b]furan-4,9-dione;
2-acetyl-6-chloro-8-hydroxynaphtho[2,3-b]furan-4,9-dione;
2-acetyl-7-chloro-8-hydroxynaphtho[2,3-b]furan-4,9-dione;
2-acetyl-8-chloro-7-hydroxynaphtho[2,3-b]furan-4,9-dione;
2-acetyl-6-chloro-7-hydroxynaphtho[2,3-b]furan-4,9-dione;
2-acetyl-5-chloro-7-hydroxynaphtho[2,3-b]furan-4,9-dione;
2-acetyl-5-chloro-6-hydroxynaphtho[2,3-b]furan-4,9-dione;
2-acetyl-7-chloro-6-hydroxynaphtho[2,3-b]furan-4,9-dione; and
2-acetyl-8-chloro-6-hydroxynaphtho[2,3-b]furan-4,9-dione.

20. A pharmaceutical composition comprising a compound according to claim 1 or a pharmaceutically acceptable salt thereof.

21. An anticancer agent comprising a compound according to claim 1 or a pharmaceutically acceptable salt thereof as an active ingredient.

22. The anticancer agent according to claim 21, wherein the cancer is at least one cancer selected from the group consisting of acute leukemia, chronic lymphocytic leukemia, chronic myelocytic leukemia, polycythemia vera, malignant lymphoma, brain tumor, head and neck cancer, esophageal cancer, thyroid cancer, small cell lung cancer, non-small-cell lung cancer, breast cancer, gastric cancer, gallbladder⋅bile duct cancer, hepatoma, pancreatic cancer, colon cancer, rectal cancer, chorioepithelioma, chorioblastoma, choriocarcinoma, endometrial cancer, cervical cancer, urothelial cancer, renal cell cancer, testicular tumor, Wilms' tumor, skin cancer, malignant melanoma, neuroblastoma, osteosarcoma, Ewing's sarcoma, and soft tissue sarcoma.

23. A method for treating a cancer, characterized by administering a therapeutically effective amount of a compound according to claim 1 or a pharmaceutically acceptable salt thereof to a patient in need of the treatment.

24. The method for treating according to claim 23, wherein the cancer is at least one cancer selected from the group consisting of acute leukemia, chronic lymphocytic leukemia, chronic myelocytic leukemia, polycythemia vera, malignant lymphoma, brain tumor, head and neck cancer, esophageal cancer, thyroid cancer, small cell lung cancer, non-small-cell lung cancer, breast cancer, gastric cancer, gallbladder⋅bile duct cancer, hepatoma, pancreatic cancer, colon cancer, rectal cancer, chorioepithelioma, chorioblastoma, choriocarcinoma, endometrial cancer, cervical cancer, urothelial cancer, renal cell cancer, testicular tumor, Wilms' tumor, skin cancer, malignant melanoma, neuroblastoma, osteosarcoma, Ewing's sarcoma, and soft tissue sarcoma.

25. The use of a compound according to claim 1 or a pharmaceutically acceptable salt thereof for the manufacture of a therapeutic agent for cancer.

26. The use of a compound according to claim 25 or a pharmaceutically acceptable salt thereof, wherein the cancer is at least one cancer selected from the group consisting of acute leukemia, chronic lymphocytic leukemia, chronic myelocytic leukemia, polycythemia vera, malignant lymphoma, brain tumor, head and neck cancer, esophageal cancer, thyroid cancer, small cell lung cancer, non-small-cell lung cancer, breast cancer, gastric cancer, gallbladder⋅bile duct cancer, hepatoma, pancreatic cancer, colon cancer, rectal cancer, chorioepithelioma, chorioblastoma, choriocarcinoma, endometrial cancer, cervical cancer, urothelial cancer, renal cell cancer, testicular tumor, Wilms' tumor, skin cancer, malignant melanoma, neuroblastoma, osteosarcoma, Ewing's sarcoma, and soft tissue sarcoma.
